(19) United States
(12) Patent Application Publication
(10) Pub. No.: US 2023/0372299 A1
(43) Pub. Date: Nov. 23, 2023

(54) PREPARATION OF TEREVALEFIM AND FORMULATIONS THEREOF

(71) Applicant: Angion Biomedica Corp., Newton, MA (US)

(72) Inventors: David Howard Upchurch, Burlingame, CA (US); An-Hu Li, Commack, NY (US); Weizhong Cai, Dix Hills, NY (US); Marc W. Andersen, Raleigh, NC (US); Khawla Abdullah Abu-Izza, Berkeley, CA (US)

(21) Appl. No.: 18/031,394

(22) PCT Filed: Oct. 13, 2021
(86) PCT No.: PCT/US2021/054776

§ 371 Date: Apr. 12, 2023

Related U.S. Application Data
(60) Provisional application No. 63/091,747, filed on Oct. 14, 2020.

(51) Int. Cl. A61K 31/4155; A61K 47/10; A61K 47/26; A61K 9/00

(52) U.S. Cl. CPC A61K 31/4155; A61K 47/10; A61K 47/26; A61K 9/0019

(57) ABSTRACT

The present disclosure provides formulations of an HGF/SF mimetic, terevalefim, as well as methods of preparing and using the formulations. The present disclosure also provides methods of synthesizing terevalefim.

### RELATED APPLICATIONS

This application claims priority to U.S. Provisional Patent Application No. 63/091,747, filed Oct. 14, 2020, the entire contents of which are hereby incorporated by reference.

### BACKGROUND

Scatter factor (SF; also known as hepatocyte growth factor (HGF), and hereinafter referred to and abbreviated as HGF/SF) is a pleiotropic growth factor that stimulates cell growth, cell motility, morphogenesis and angiogenesis. HGF/SF is produced as an inactive monomer (˜100 kDa) which is proteolytically converted to its active form. Active HGF/SF is a heparin-binding heterodimeric protein composed of a 62 kDa α chain and a 34 kDa β chain. HGF/SF has a short half-life of 3-5 min (Chang, H.-K., et al., Mol Ther. 2016 September; 24(9): 1644-1654). HGF/SF is a potent mitogen for parenchymal liver, epithelial and endothelial cells (Matsumoto, K, and Nakamura, T., 1997, Biochem. Biophys. Res. Commun. 239, 639-44; Boros, P. and Miller, C. M., 1995, Lancet 345, 293-5). It stimulates the growth of endothelial cells and also acts as a survival factor against endothelial cell death (Morishita, R, et al., 1997, Diabetes 46:138-42). HGF/SF synthesized and secreted by vascular smooth muscle cells stimulates endothelial cells to proliferate, migrate and differentiate into capillary-like tubes in vitro (Grant, D. S, et al., 1993, Proc. Natl. Acad. Sci. USA 90:1937-41; Morishita, R., et al., 1999, Hypertension 33:1379-84). HGF/SF-containing implants in mouse subcutaneous tissue and rat cornea induce growth of new blood vessels from surrounding tissue. HGF/SF protein is expressed at sites of neovascularization including in tumors (Jeffers, M., et al., 1996, J. Mol. Med. 74:505-13; Moriyama, T., et al., 1999, Int. J. Mol. Med. 3:531-6). These findings suggest that HGF/SF plays a significant role in the formation and repair of blood vessels under physiological and pathological conditions.

### SUMMARY

The present disclosure provides methods of preparing compounds useful as HGF/SF mimetics, such as terevalefim. The present disclosure also provides synthetic intermediates and compositions thereof useful in the preparation of said HGF/SF mimetics, such as terevalefim.

The present disclosure also provides certain liquid formulations of HGF/SF mimetics, such as terevalefim, suitable for intravenous administration. In some embodiments, the present disclosure provides formulations which achieve certain desirable characteristics, e.g., particular concentrations of active agent, homogeneity, particular impurity profiles, stable to storage, etc.

In some embodiments, the present disclosure provides methods of preparing liquid formulations of HGF/SF mimetics, such as terevalefim, e.g., on an industrially relevant scale and/or in a manner suitable for administration to humans.

The present disclosure also provides methods of using terevalefim and provided formulations thereof.

### BRIEF DESCRIPTION OF THE DRAWINGS

FIG. 1 provides XRPD pattern of Terevalefim Lot I.

FIG. 2 provides TGA curve of Terevalefim Lot I.

FIG. 3 provides DSC thermogram of Terevalefim Lot I.

FIG. 4 provides XRPD pattern of terevalefim Form A calculated from single crystal X-ray diffraction data.

FIG. 5 provides XRPD pattern of terevalefim Form A.

FIG. 6 provides TGA curve of terevalefim Form A.

FIG. 7 provides DSC thermogram of terevalefim Form A.

FIG. 8 provides a comparison of XRPD patterns of Terevalefim Lot I and terevalefim Form A.

FIG. 9 provides XRPD pattern of terevalefim Form C calculated from single crystal X-ray diffraction data.

FIG. 10 provides XRPD pattern of terevalefim Form C.

FIG. 11 provides TGA curve of terevalefim Form C.

FIG. 12 provides DSC thermogram of terevalefim Form C.

FIG. 13 provides XRPD pattern of terevalefim Form D calculated from single crystal X-ray diffraction data.

FIG. 14 provides XRPD pattern of terevalefim Form D.

FIG. 15 provides TGA curve of terevalefim Form D.

FIG. 16 provides DSC thermogram of terevalefim Form D.

FIG. 17A provides single crystal X-ray crystallography of terevalefim Form A. N and S atoms are labeled; unlabeled non-hydrogen atoms are carbon.

FIG. 17B provides single crystal X-ray crystallography of terevalefim Form C. N, S, and O atoms are labeled; unlabeled non-hydrogen atoms are carbon.

FIG. 17C provides single crystal X-ray crystallography of terevalefim Form D. N, S, and O atoms are labeled; unlabeled non-hydrogen atoms are carbon.

### DETAILED DESCRIPTION

### Definitions

The term “about”, when used herein in reference to a value, refers to a value that is similar, in context to the referenced value. In general, those skilled in the art, familiar with the context, will appreciate the relevant degree of variance encompassed by “about” in that context. In some embodiments, the term “about” refers to ±10% of a given value.

As used herein, the term “administering” or “administration” typically refers to the administration of a composition to a subject to achieve delivery of an active agent to a site of interest (e.g., a target site which may, in some embodiments, be a site of disease or damage, and/or a site of responsive processes, cells, tissues, etc.) As will be understood by those skilled in the art, reading the present disclosure, in some embodiments, one or more particular routes of administration may be feasible and/or useful in the practice of the present disclosure. For example, in some embodiments, administration may be parenteral. In some embodiments, administration may be oral. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time. As described herein, in many embodiments, administration is parenteral, e.g., via intravenous (IV) administration, which in some embodiments may be or comprise IV perfusion); in some embodiments, one or more instances of perfusion may be performed. In some embodiments, amount perfused and/or rate of perfusion may be selected, for example, in light of a characteristic such as subject weight, age, presence and/or extent of one or more relevant symptom(s), timing relative to transplant procedure, etc.

As used herein, the term “comparable” refers to two or more agents, entities, situations, sets of conditions, circumstances, individuals, or populations, etc., that may not be identical to one another but that are sufficiently similar to permit comparison there between so that one skilled in the art will appreciate that conclusions may reasonably be drawn based on differences or similarities observed. In some embodiments, comparable agents, entities, situations, sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, circumstances, individuals, or populations, etc. to be considered comparable. For example, those of ordinary skill in the art will appreciate that sets of circumstances, agents, entities, situations, individuals, or populations are comparable to one another when characterized by a sufficient number and type of substantially identical features to warrant a reasonable conclusion that differences in results obtained or phenomena observed under or with different agents, entities, situations, sets of circumstances, individuals, or populations are caused by or indicative of the variation in those features that are varied.

As used herein, the term “pharmaceutical composition” refers to a composition comprising a pharmaceutical active (which may be, comprise, or otherwise become an active agent upon administration of the composition), formulated together with one or more pharmaceutically acceptable carriers. In some embodiments, a pharmaceutical composition is or comprises a pharmaceutical active present in a unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces. In some embodiments, as described herein, a pharmaceutical composition is formulated for parenteral administration (e.g., for IV administration such as by infusion).

The term “pharmaceutically acceptable salt form,” as used herein, refers to a form of a relevant compound as a salt appropriate for use in pharmaceutical contexts, i.e., salts which are, within the scope of sound medical judgment, suitable for use in contact with the tissues of humans and/or animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977).

As used herein, the term “subject” refers to an organism, typically a mammal (e.g., a human). In some embodiments, a subject is suffering from a relevant disease, disorder or condition. In some embodiments, a human subject is an adult, adolescent, or pediatric subject. In some embodiments, a subject is at risk of (e.g., susceptible to), e.g., at elevated risk of relative to an appropriate control individual or population thereof, a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is an individual to whom diagnosis and/or therapy and/or prophylaxis is and/or has been administered. The terms “subject” and “patient” are used interchangeably herein.

As used herein, the term “treat” (also “treatment” or “treating”) refers to any administration of a therapy that partially or completely alleviates, ameliorates, relieves, inhibits, delays onset of, reduces severity of, and/or reduces incidence of one or more symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition.

### Hepatocyte Growth Factor Mimetics

PCT Application No. PCT/US2003/040917, filed Dec. 19, 2003 and published as WO2004/058721 on Jul. 15, 2004, the entirety of which is hereby incorporated by reference, describes certain compounds that act as HGF/SF mimetics. Such compounds include terevalefim:

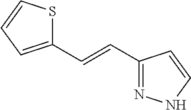 [SMILES: C(=C\c1cccs1)/c1cc[nH]n1]

or a pharmaceutically acceptable salt thereof (i.e., terevalefim in a pharmaceutically acceptable salt form).

Terevalefim has been demonstrated to be remarkably useful for treatment of a variety of conditions including, for example, fibrotic liver disease, ischemia-reperfusion injury, cerebral infarction, ischemic heart disease, renal disease, lung fibrosis, damaged and/or ischemic organs, transplants or grafts, stroke, cerebrovascular disease, and renal fibrosis, among others (see, for example, WO 2004/058721, WO 2010/005580, US 2011/0230407, U.S. Pat. No. 7,879,898, and WO 2009/064422, each of which is hereby incorporated by reference.) Exemplary methods of using terevalefim for, e.g., treating delayed graft function after kidney transplantation and acute lung injury, are described in WO 2021/087392 and WO 2021/183774, each of which is hereby incorporated by reference. In particular, terevalefim is or has been the subject of clinical trials for delayed graft function in recipients of a deceased donor kidney (Clinicaltrials.gov identifier: NCT02474667), acute kidney injury after cardiac surgery involving cardiopulmonary bypass (Clinicaltrials.gov identifier: NCT02771509), and COVID-19 pneumonia (Clinicaltrials.gov identifier: NCT04459676). Without wishing to be bound by any particular theory, it is believed that terevalefim's HGF mimetic capability imparts a variety of beneficial attributes and activities.

Terevalefim has a CAS Registry No. of 1070881-42-3 and is also known by at least the following names:
3-[(1E)-2-(thiophen-2-yl)ethen-1-yl]-1H-pyrazole; and
(E)-3-[2-(2-thienyl)vinyl]-1H-pyrazole.

Those skilled in the art will appreciate that terevalefim has a structure that can exist in various tautomeric forms, including (E)-3-[2-(2-thienyl)vinyl]-1H-pyrazole and (E)-5-[2-(2-thienyl)vinyl]-1H-pyrazole, or any mixture thereof. Moreover, those skilled in the art, reading the present disclosure, will appreciate that, in many embodiments, teachings described herein are not limited to any particular tautomeric form. The present disclosure contemplates use of all tautomeric forms of terevalefim.

In some embodiments, terevalefim is provided and/or utilized (e.g., for inclusion in a composition and/or for delivery to a subject) in accordance with the present disclosure in a form such as a salt form. As already noted herein, pharmaceutically acceptable salts are well known in the art.

In some embodiments, terevalefim is provided and/or utilized (e.g., for inclusion in (e.g., during one or more steps of manufacturing of) a composition and/or for delivery to a subject) in accordance with the present disclosure in a form such as a solid form. In some embodiments, terevalefim is provided and/or utilized in accordance with the present disclosure in an amorphous solid form, in a crystalline solid form, or in a mixture thereof. In some embodiments, a composition is substantially free of amorphous terevalefim. As used herein, the term “substantially free” means lacking a significant amount (e.g., less than about 10%, less than about 5%, less than about 3%, less than about 2%, or less than about 1%). In some embodiments, a composition comprises at least about 90% by weight of crystalline terevalefim. In some embodiments, a composition comprises at least about 95% by weight of crystalline terevalefim. In some embodiments, a composition comprises at least about 97%, about 98%, or about 99% by weight of crystalline terevalefim. In some embodiments, a crystalline solid form may be or comprise a solvate, hydrate, or an unsolvated form. The use of any and all such forms are contemplated by the present disclosure.

Terevalefim can exist in at least three distinct crystalline solid forms, designated herein as Form A, Form C, and Form D. Exemplary methods of preparing each of these crystalline solid forms is provided in the Examples section.

In some embodiments, a crystalline solid form of terevalefim is Form A. In some embodiments, Form A of terevalefim is unsolvated (e.g., anhydrous).

In some embodiments, Form A is characterized by one or more peaks in its XRPD pattern selected from those at about 8.64, about 11.04, about 17.34, about 25.06, and about 25.70 degrees 2-theta. In some embodiments, Form A is characterized by two or more peaks in its XRPD pattern selected from those at about 8.64, about 11.04, about 17.34, about 25.06, and about 25.70 degrees 2-theta. In some embodiments, Form A is characterized by three or more peaks in its XRPD pattern selected from those at about 8.64, about 11.04, about 17.34, about 25.06, and about 25.70 degrees 2-theta.

In some embodiments, Form A is characterized by peaks in its XRPD pattern at about 8.64, about 11.04, about 17.34, about 25.06, and about 25.70 degrees 2-theta. In some embodiments, Form A is characterized by peaks in its XRPD pattern at about 8.64, about 11.04, about 17.34, about 25.06, and about 25.70 degrees 2-theta, corresponding to d-spacing of about 10.22, about 8.01, about 5.11, about 3.55, and about 3.46 angstroms.

In some embodiments, Form A is characterized by substantially all of the peaks (degrees 2-theta) in its XRPD pattern, optionally corresponding to d-spacing (angstroms), at about:

| 2θ (°) | d-spacing (Å) |
|---|---|
| 8.64 | 10.22 |
| 11.04 | 8.01 |
| 11.67 | 7.57 |
| 16.06 | 5.51 |
| 17.34 | 5.11 |
| 18.27 | 4.85 |
| 18.69 | 4.74 |
| 19.49 | 4.55 |
| 20.66 | 4.30 |
| 21.09 | 4.21 |
| 21.70 | 4.09 |
| 22.10 | 4.02 |
| 22.76 | 3.90 |
| 23.46 | 3.79 |
| 23.74 | 3.74 |
| 25.06 | 3.55 |
| 25.70 | 3.46 |
| 26.12 | 3.41 |
| 26.32 | 3.38 |
| 27.64 | 3.23 |
| 27.78 | 3.21 |
| 28.31 | 3.15 |
| 28.49 | 3.13 |
| 29.04 | 3.07 |
| 29.95 | 2.98 |
| 31.59 | 2.83 |
| 31.82 | 2.81 |
| 32.25 | 2.77 |
| 33.22 | 2.69 |
| 34.21 | 2.62 |
| 34.42 | 2.60 |
| 35.08 | 2.56 |
| 35.53 | 2.52 |
| 36.33 | 2.47 |
| 36.70 | 2.45 |
| 37.16 | 2.42 |
| 37.65 | 2.39 |
| 39.02 | 2.31 |
| 39.60 | 2.27 |
| 39.81 | 2.26 |

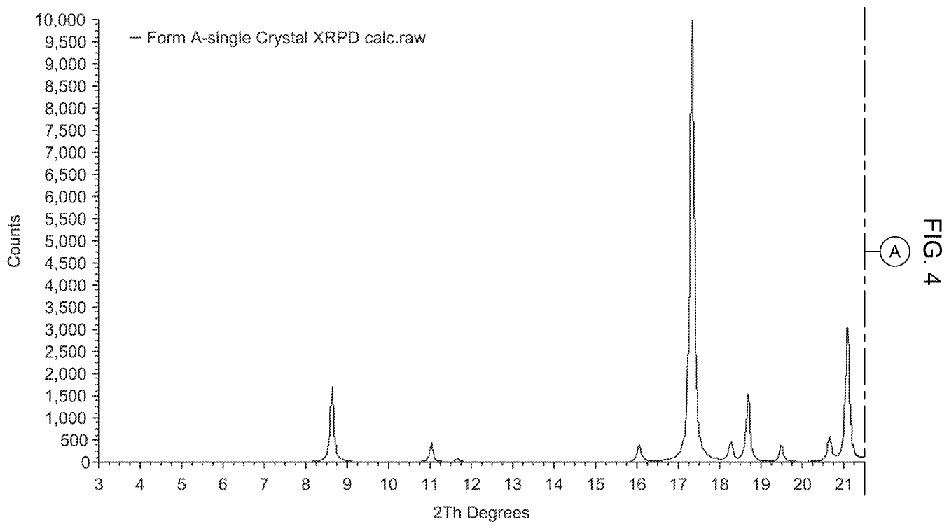

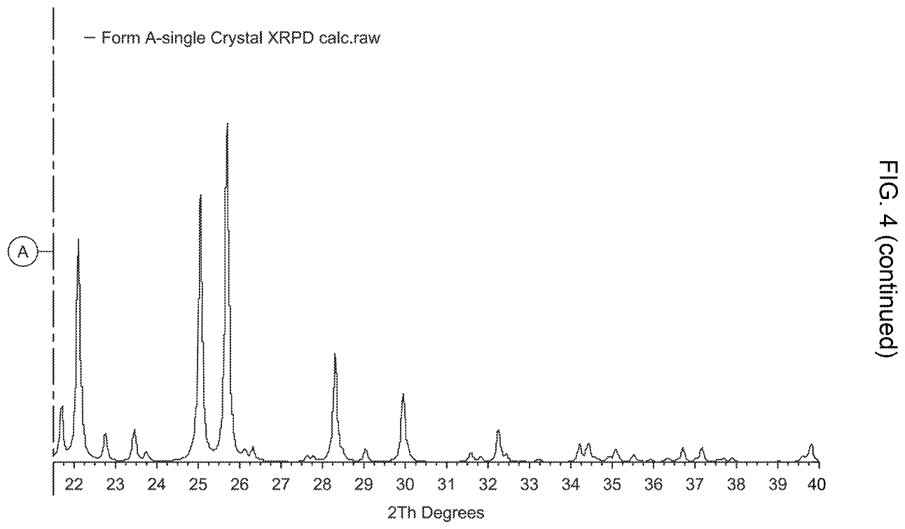

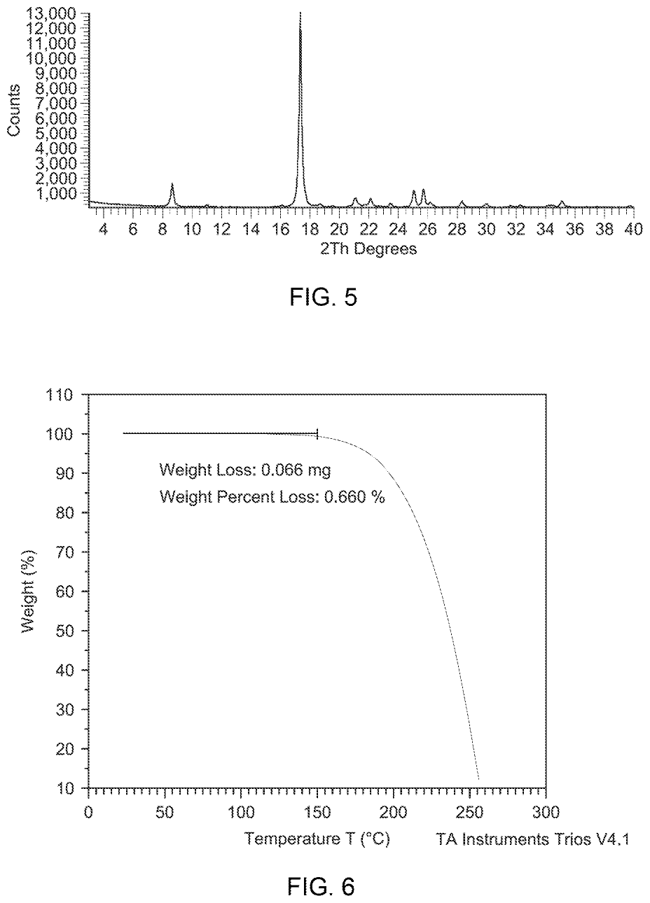

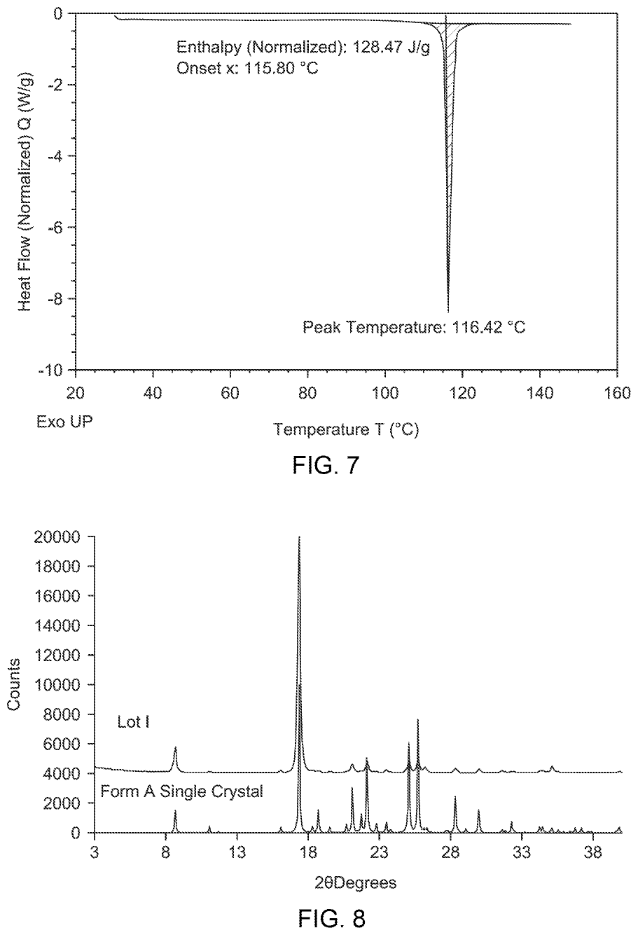

In some embodiments, Form A is characterized by one or more of the following:
(i) an XRPD pattern substantially similar to that depicted in FIG. 4 and/or FIG. 5;
(ii) a TGA pattern substantially similar to that depicted in FIG. 6;
(iii) a DSC pattern substantially similar to that depicted in FIG. 7; and
(iv) a melting point of about 116.42° C.

In some embodiments, a crystalline solid form of terevalefim is Form C. In some embodiments, Form C of terevalefim is a propylene glycol solvate. As used herein, the term “solvate” refers to a solid form with a stoichiometric amount of one or more solvents (e.g., water, ethylene glycol, propylene glycol, etc.) incorporated into the crystal structure. For example, a solvated or heterosolvated polymorph can comprise 0.05, 0.1, 0.2, 0.5, 1.0, 1.5, 2.0, etc. equivalents independently of one or more solvents incorporated into the crystal lattice. In some embodiments, Form C of terevalefim is a propylene glycol solvate, wherein the ratio of terevalefim to propylene glycol is about 2:1.

In some embodiments, Form C is characterized by one or more peaks in its XRPD pattern selected from those at about 11.88, about 17.50, about 19.08, about 21.28, and about 23.07 degrees 2-theta. In some embodiments, Form C is characterized by two or more peaks in its XRPD pattern selected from those at about 11.88, about 17.50, about 19.08, about 21.28, and about 23.07 degrees 2-theta. In some embodiments, Form C is characterized by three or more peaks in its XRPD pattern selected from those at about 11.88, about 17.50, about 19.08, about 21.28, and about 23.07 degrees 2-theta.

In some embodiments, Form C is characterized by peaks in its XRPD pattern at about 11.88, about 17.50, about 19.08, about 21.28, and about 23.07 degrees 2-theta. In some embodiments, Form C is characterized by peaks in its XRPD pattern at about 11.88, about 17.50, about 19.08, about 21.28, and about 23.07 degrees 2-theta, corresponding to d-spacing of about 7.44, about 5.06, about 4.65, about 4.17, and about 3.85 angstroms.

In some embodiments, Form C is characterized by substantially all of the peaks (degrees 2-theta) in its XRPD pattern, optionally corresponding to d-spacing (angstroms), at about:

| 2θ (°) | d-spacing (Å) |
|---|---|
| 6.20 | 14.24 |
| 11.88 | 7.44 |
| 12.41 | 7.13 |
| 14.79 | 5.99 |
| 15.72 | 5.63 |
| 17.50 | 5.06 |
| 17.78 | 4.98 |
| 19.08 | 4.65 |
| 19.36 | 4.58 |
| 21.28 | 4.17 |
| 21.89 | 4.06 |
| 23.07 | 3.85 |
| 23.69 | 3.75 |
| 23.92 | 3.72 |
| 24.70 | 3.60 |
| 24.90 | 3.57 |
| 25.50 | 3.49 |
| 25.71 | 3.46 |
| 26.28 | 3.39 |
| 27.74 | 3.21 |
| 28.13 | 3.17 |
| 29.10 | 3.07 |
| 29.31 | 3.04 |
| 29.55 | 3.02 |
| 30.51 | 2.93 |
| 31.15 | 2.87 |
| 31.76 | 2.82 |
| 32.93 | 2.72 |
| 33.19 | 2.70 |
| 33.36 | 2.68 |
| 34.63 | 2.59 |
| 35.05 | 2.56 |
| 35.40 | 2.53 |
| 35.90 | 2.50 |
| 36.20 | 2.48 |
| 36.60 | 2.45 |
| 37.57 | 2.39 |
| 37.69 | 2.38 |
| 38.05 | 2.36 |
| 38.54 | 2.33 |
| 38.80 | 2.32 |
| 39.05 | 2.30 |
| 39.46 | 2.28 |
| 39.82 | 2.26 |

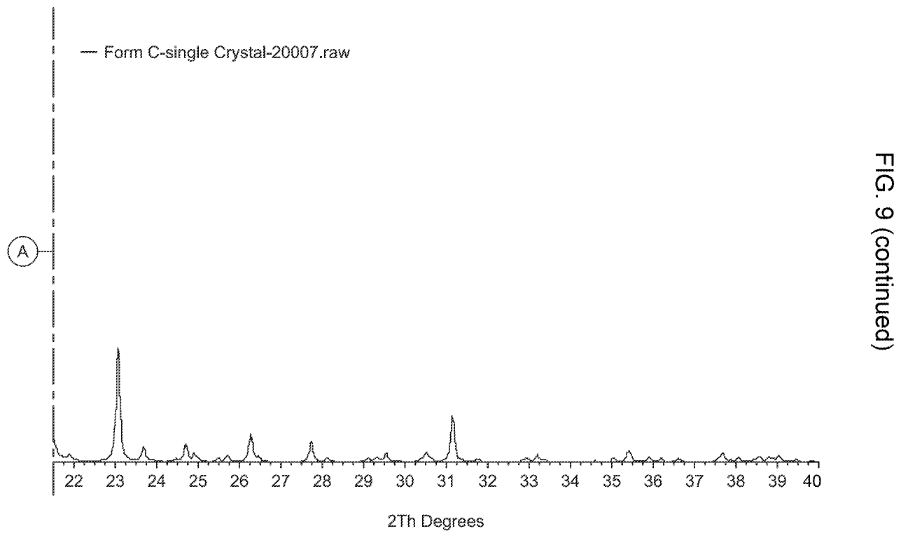

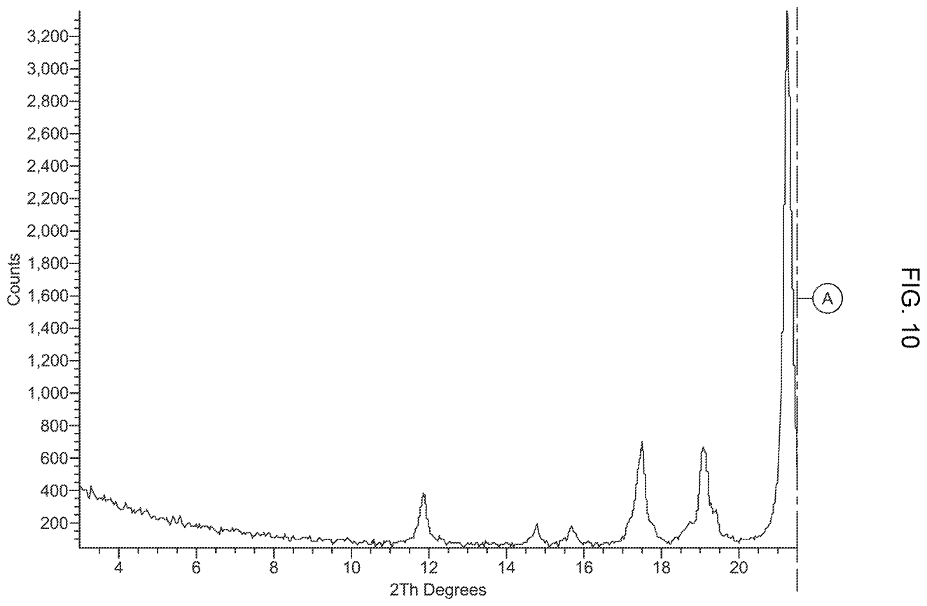

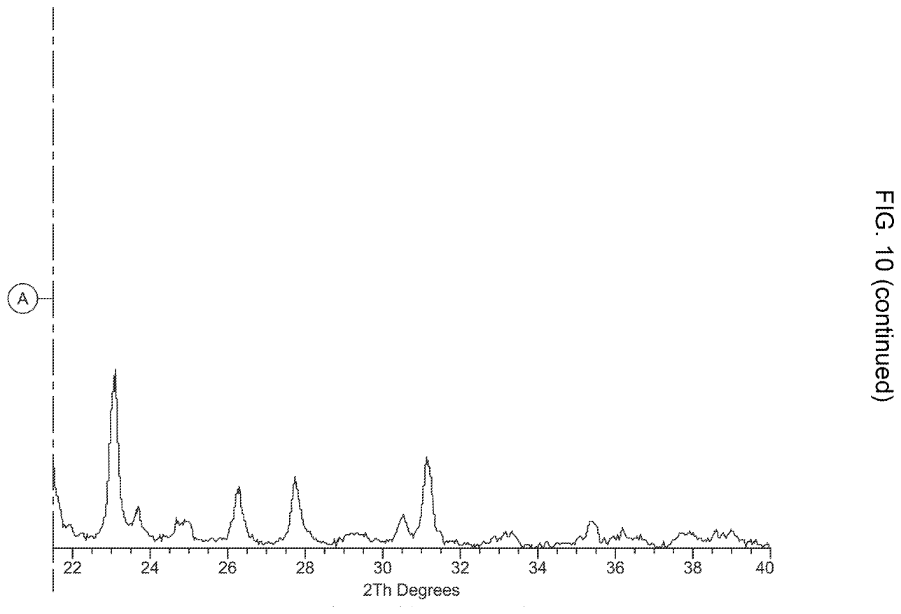

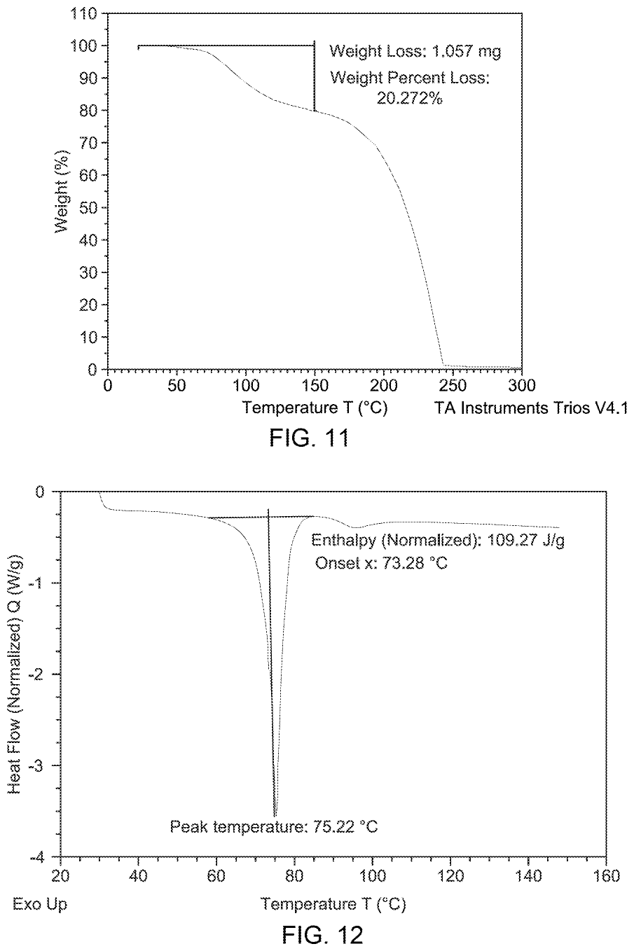

In some embodiments, Form C of terevalefim has one or more of the following characteristics:
(i) an XRPD pattern substantially similar to that depicted in FIG. 9 and/or FIG. 10,
(ii) a TGA pattern substantially similar to that depicted in FIG. 11;
(iii) a DSC pattern substantially similar to that depicted in FIG. 12; and
(iv) a melting point of about 75.22° C.

In some embodiments, a crystalline solid form of terevalefim is Form D. In some embodiments, Form D of terevalefim is an ethylene glycol solvate. In some embodiments, Form D of terevalefim is an ethylene glycol solvate, wherein the ratio of terevalefim to ethylene glycol is about 2:1.

In some embodiments, Form D is characterized by one or more peaks in its XRPD pattern selected from those at about 12.28, about 15.10, about 18.06, about 21.58, and about 23.88 degrees 2-theta. In some embodiments, Form D is characterized by two or more peaks in its XRPD pattern selected from those at about 12.28, about 15.10, about 18.06, about 21.58, and about 23.88 degrees 2-theta. In some embodiments, Form D is characterized by three or more peaks in its XRPD pattern selected from those at about 12.28, about 15.10, about 18.06, about 21.58, and about 23.88 degrees 2-theta.

In some embodiments, Form D is characterized by peaks in its XRPD pattern at about 12.28, about 15.10, about 18.06, about 21.58, and about 23.88 degrees 2-theta. In some embodiments, Form D is characterized by peaks in its XRPD pattern at about 12.28, about 15.10, about 18.06, about 21.58, and about 23.88 degrees 2-theta, corresponding to d-spacing of about 7.20, about 5.86, about 4.91, about 4.11, and about 3.72 angstroms.

In some embodiments, Form D is characterized by substantially all of the peaks (degrees 2-theta) in its XRPD pattern, optionally corresponding to d-spacing (angstroms), at about:

| 2θ (°) | d-spacing (Å) |
|---|---|
| 6.19 | 14.28 |
| 12.28 | 7.20 |
| 12.38 | 7.14 |
| 15.10 | 5.86 |
| 15.24 | 5.81 |
| 16.17 | 5.48 |
| 17.60 | 5.03 |
| 18.06 | 4.91 |
| 18.63 | 4.76 |
| 19.11 | 4.64 |
| 19.44 | 4.56 |
| 20.35 | 4.36 |
| 21.58 | 4.11 |
| 21.95 | 4.05 |
| 22.55 | 3.94 |
| 23.88 | 3.72 |
| 24.70 | 3.60 |
| 24.88 | 3.58 |
| 25.57 | 3.48 |
| 25.76 | 3.46 |
| 26.07 | 3.42 |
| 26.33 | 3.38 |
| 26.50 | 3.36 |
| 26.75 | 3.33 |
| 26.99 | 3.30 |
| 27.25 | 3.27 |
| 28.51 | 3.13 |
| 28.72 | 3.11 |
| 29.96 | 2.98 |
| 30.10 | 2.97 |
| 30.26 | 2.95 |
| 30.47 | 2.93 |
| 30.62 | 2.92 |
| 30.75 | 2.90 |
| 31.27 | 2.86 |
| 31.57 | 2.83 |
| 31.83 | 2.81 |
| 31.97 | 2.80 |
| 32.38 | 2.76 |
| 32.67 | 2.74 |
| 33.61 | 2.66 |
| 33.82 | 2.65 |
| 34.84 | 2.57 |
| 35.78 | 2.51 |
| 36.02 | 2.49 |
| 36.36 | 2.47 |
| 36.53 | 2.46 |
| 36.66 | 2.45 |
| 37.10 | 2.42 |
| 37.44 | 2.40 |
| 37.81 | 2.38 |
| 38.38 | 2.34 |
| 38.72 | 2.32 |
| 39.24 | 2.29 |
| 39.78 | 2.26 |

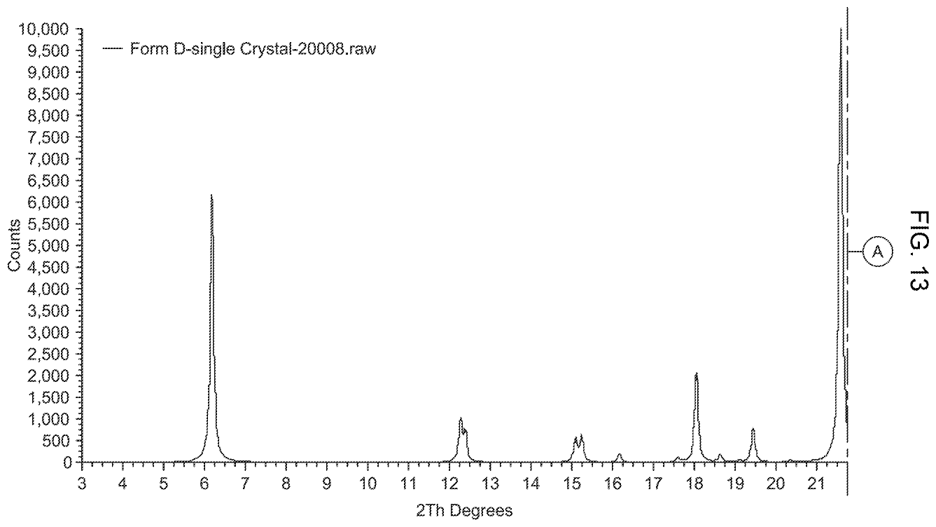

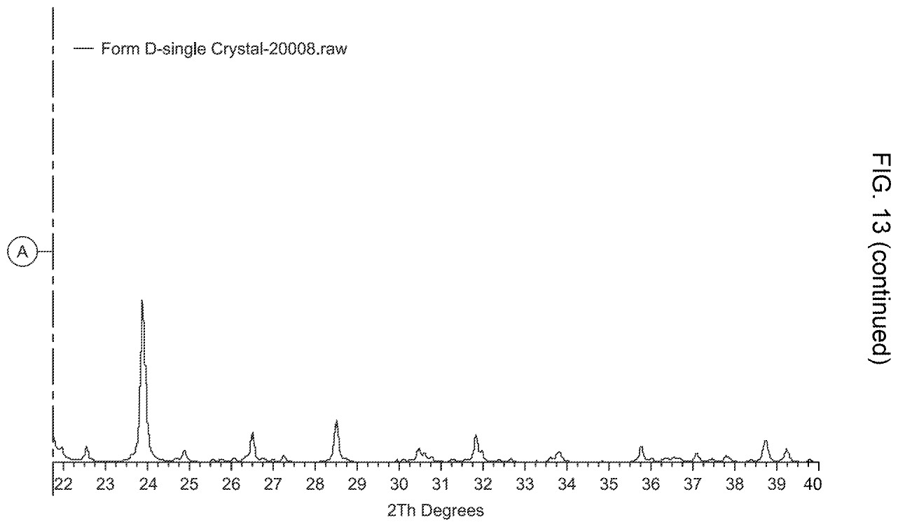

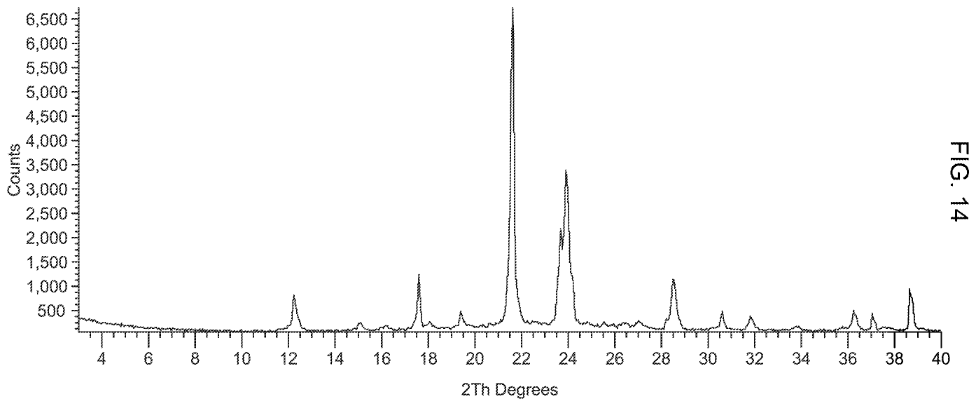

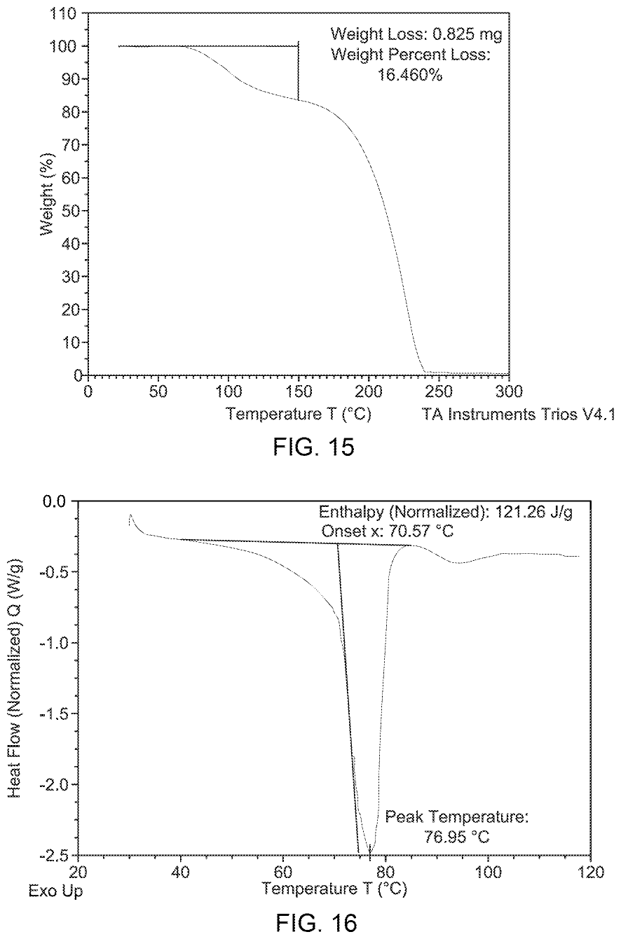

In some embodiments, Form D of terevalefim has one or more of the following characteristics:
(i) an XRPD pattern substantially similar to that depicted in FIG. 13 and/or FIG. 14;
(ii) a TGA pattern substantially similar to that depicted in FIG. 15;
(iii) a DSC pattern substantially similar to that depicted in FIG. 16; and
(iv) a melting point of about 76.95° C.

As used herein, the term “about” when used in reference to a degree 2-theta value refers to the stated value±0.2 degree 2-theta. In some embodiments, “about” refers to the stated value±0.1 degree 2-theta.

Unless otherwise indicated, as used herein “terevalefim” refers to (E)-3-[2-(2-thienyl)vinyl]-1H-pyrazole in any available form, such as, e.g., a tautomer, salt form, and/or solid form thereof.

### Synthesis of Terevalefim

In some embodiments, the present disclosure provides methods for preparing compounds useful as HGF/SF mimetics, such as terevalefim. A synthesis of terevalefim is described in detail in Example 7 of WO 2004/058721 (“the '721 Synthesis”). The '721 Synthesis is depicted in Scheme 1:

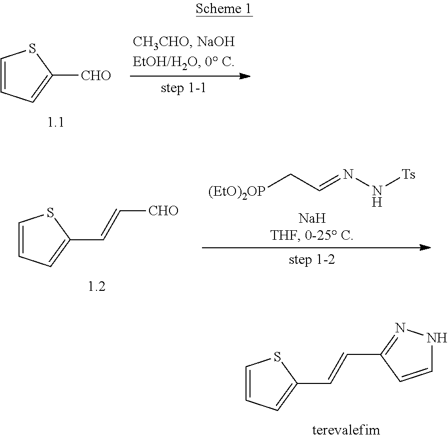

The '721 Synthesis includes certain features which are not desirable for preparation of terevalefim at scale and/or with consistency and/or with suitable purity for use in humans. For example, the '721 Synthesis includes preparation of aldehyde compound 1.2, a viscous oil that is difficult to purify with standard techniques. Additionally, the '721 Synthesis uses a diethoxyphosphorylacetaldehyde tosylhydrazone reagent in step 1-2. As such, step 1-2 has poor atom economy and results in multiple byproducts that must be purified away from the final product of terevalefim. Step 1-2 also uses sodium hydride, a highly reactive base that can be difficult to control and often results in byproducts that must be purified away from the final product of terevalefim. Such purification steps can be costly and time-consuming. In some embodiments, the present disclosure encompasses the recognition that one or more features of the '721 Synthesis can be improved to increase yield and/or increase reliability and/or increase scale and/or reduce byproducts. In some embodiments, the present disclosure provides such a synthesis, as detailed herein.

In some embodiments, the present disclosure provides a synthesis of terevalefim as depicted in Scheme 2:

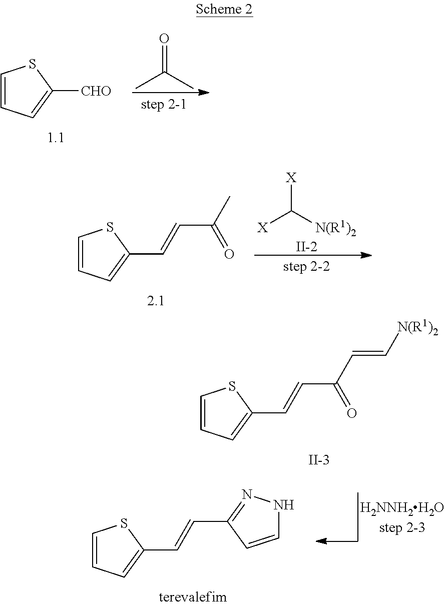

wherein X and R¹ are defined below and in classes and subclasses as described herein.

It will be appreciated that compounds described herein, e.g., compounds in Scheme 2, may be provided and/or utilized in a salt form. For example, compounds which contain a basic nitrogen atom may form a salt with a suitable acid. Alternatively and/or additionally, compounds which contain an acidic moiety, such as a carboxylic acid group, may form a salt with a suitable base. Suitable counterions are well known in the art, e.g., see generally, March's Advanced Organic Chemistry: Reactions, Mechanisms, and Structure, M. B. Smith and J. March, 5^{th} Edition, John Wiley & Sons, 2001. All forms of the compounds in Scheme 2 are contemplated by and within the scope of the present disclosure.

### Step 2-1 of Scheme 2

Step 2-1 comprises a condensation-elimination reaction between commercially available thiophene-2-carboxaldehyde (1.1) and acetone to provide an α,β-unsaturated ketone compound (2.1).

In some embodiments, the present disclosure provides a method comprising steps of:
(i) providing compound 1.1:

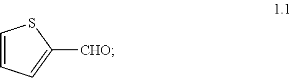

and
(ii) contacting compound 1.1 with acetone in the presence of a suitable base, to provide compound 2.1:

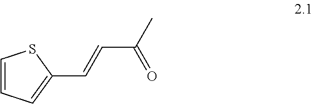

In some embodiments, a suitable base in Step 2-1 is an inorganic base. In some embodiments, a suitable base in Step 2-1 is a hydroxide base or an alkoxide base. In some embodiments, a suitable base in Step 2-1 is LiOR, NaOR, or KOR, wherein R is hydrogen or C₁₋₆ alkyl. In some embodiments, a suitable base in Step 2-1 is a hydroxide base such as LiOR, NaOR, or KOR, wherein R is hydrogen. In some embodiments, a suitable base in Step 2-1 is NaOH. In some embodiments, a suitable base in Step 2-1 is an alkoxide base such as LiOR, NaOR, or KOR, wherein R is C₁₋₆ alkyl (e.g., wherein R is methyl, ethyl, or tert-butyl).

In some embodiments, Step 2-1 is performed in a suitable solvent. In some such embodiments, a suitable solvent is or comprises a mixture of acetone and an aqueous or alcoholic solvent. In some embodiments, a suitable solvent is or comprises acetone. In some embodiments, a suitable solvent is or comprises water. In some embodiments, a suitable solvent is or comprises methanol or ethanol. In some embodiments, a suitable solvent is a mixture of acetone and water.

In some embodiments, Step 2-1 comprises cooling a mixture of compound 1.1 and acetone to a temperature, e.g., between about 0° C. and about 10° C., e.g., about 5° C. In some embodiments, Step 2-1 comprises allowing the reaction mixture to warm to a temperature between about 15° C. and about 25° C., e.g., about 20° C. (e.g., after adding the solution of suitable base). In some embodiments, Step 2-1 comprises allowing the reaction mixture to stir for a period of time (e.g., after warming it to about 20° C.), such as for about 2-3 hours.

In some embodiments, Step 2-1 comprises extracting compound 2.1 from the reaction mixture, e.g., using a suitable solvent, to provide an organic fraction comprising compound 2.1. In some such embodiments, a suitable solvent for extracting compound 2.1 is an organic solvent (e.g., dichloromethane, ethyl acetate, etc.). In some embodiments, the organic fraction is contacted with a drying agent (e.g., sodium sulfate, magnesium sulfate, etc.). In some embodiments, the organic fraction is subjected to azeotropic distillation. In some embodiments, the organic fraction is concentrated to afford compound 2.1. In some embodiments, the solvent of the organic fraction is changed from a first solvent (e.g., dichloromethane, ethyl acetate, etc.) to a second solvent (e.g., toluene). In some embodiments, changing the solvent of the organic fraction comprises adding the second solvent (e.g., toluene) and then causing the first solvent (e.g., dichloromethane, ethyl acetate, etc.) to evaporate, thereby providing a solution of compound 2.1 in the second solvent (e.g., toluene). In some embodiments, the solution of compound 2.1 in the second solvent (e.g., toluene) is azeotropically dried to remove residual water.

In some embodiments, the crude product of Step 2-1 (i.e., 2.1) is isolated as an oil (e.g., substantially free of solvent). In some embodiments, the crude product of Step 2-1 (i.e., 2.1) is purified using any suitable means, such as by fractional distillation.

In some embodiments, the crude product of Step 2-1 (i.e., 2.1) is used directly in Step 2-2 without purification and/or isolation. In some embodiments, a solution of compound 2.1 in toluene is used directly in Step 2-2 without further purification. Without wishing to be bound by any particular theory, using the crude product of Step 2-1 directly in Step 2-2 streamlines the overall synthesis of terevalefim by, e.g., reducing time, energy-intensive purification, and/or avoiding degradation of 2.1.

### Step 2-2 of Scheme 2

Step 2-2 of Scheme 2 comprises a condensation reaction between compound 2.1 and a compound of formula II-2 to provide a compound of formula II-3.

In some embodiments, the present disclosure provides a method comprising steps of:
(i) providing a compound 2.1:

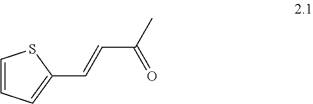

and
(ii) contacting compound 2.1 with a compound of formula II-2:

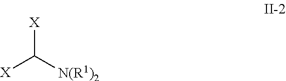

wherein each X is independently a suitable leaving group and each R¹ is independently C₁₋₆ aliphatic, under suitable conditions to provide a compound of formula II-3:

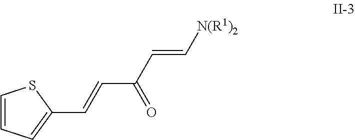

wherein each R¹ is as defined for formula II-2.

In some embodiments of a compound of formula II-2, each R¹ is independently C₁₋₆ aliphatic. In some embodiments, each R¹ is independently C₁₋₆ alkyl. In some embodiments, each R¹ is independently C₁₋₄ alkyl. In some embodiments, R¹ is methyl. In some embodiments, R¹ is ethyl. In some embodiments, R¹ is n-propyl. In some embodiments, R¹ is n-butyl.

In some embodiments of a compound of formula II-2, each X is independently a suitable leaving group. As used herein, a suitable “leaving group” is a chemical group that is readily displaced by a nucleophilic entity. Suitable leaving groups are well known in the art, e.g., see generally, March's Advanced Organic Chemistry: Reactions, Mechanisms, and Structure, M. B. Smith and J. March, 5^{th} Edition, John Wiley & Sons, 2001. Such leaving groups include, but are not limited to, halogen, optionally substituted alkoxy, optionally substituted aryloxy, optionally substituted alkylsulfonyloxy, optionally substituted alkenylsulfonyloxy, and optionally substituted arylsulfonyloxy. For the above-mentioned “optionally substituted” leaving groups, the groups may be optionally substituted with C₁₋₄ aliphatic, C₁₋₄ haloaliphatic, halogen, or nitro. In some embodiments, examples of suitable leaving groups include fluoro, chloro, bromo, iodo, mesyloxy, tosyloxy, trifyloxy, benzenesulfonyloxy, nosyloxy, brosyloxy, methoxy, ethoxy, isopropxy, and tert-butyloxy.

In some embodiments of a compound of formula II-2, each X is independently —OR² or —N(R²)₂, wherein each R² is independently C₁₋₆ aliphatic. In some embodiments, each R² is independently C₁₋₆ alkyl. In some embodiments, each R² is independently C₁₋₄ alkyl. In some embodiments, R² is methyl. In some embodiments, R² is ethyl. In some embodiments, R² is n-propyl. In some embodiments, R² is isopropyl. In some embodiments, R² is n-butyl. In some embodiments, R² is tert-butyl.

In some embodiments, X is —OR². In some embodiments, X is —OCH₃. In some embodiments, X is —OCH₂CH₃. In some embodiments, X is —OCH₂CH₂CH₃. In some embodiments, X is —OCH(CH₃)₂. In some embodiments, X is —OCH₂CH₂CH₂CH₃. In some embodiments, X is —OC(CH₃)₃.

In some embodiments, X is —N(R²)₂. In some embodiments, X is —N(CH₃)₂. In some embodiments, X is —N(CH₂CH₃)₂. In some embodiments, X is —N(CH₂CH₂CH₃)₂. In some embodiments, X is —N(CH₂CH₂CH₂CH₃)₂. In some embodiments, X is —N(C(CH₃)₃)₂.

In some embodiments, a compound of formula II-2 is selected from:

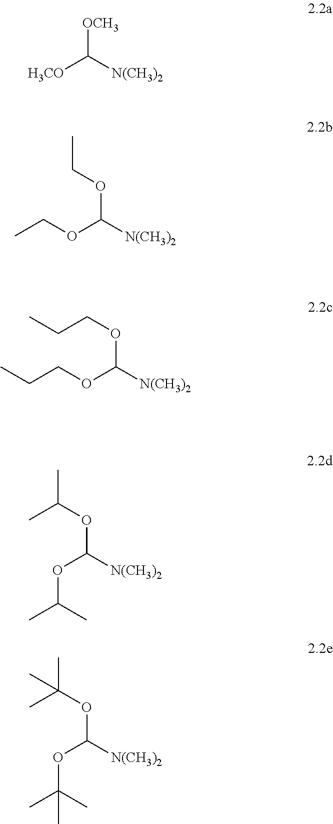

In some embodiments of a compound of formula II-3, each R¹ is independently C₁₋₆ aliphatic. In some embodiments, each R¹ is independently C₁₋₆ alkyl. In some embodiments, each R¹ is independently C₁₋₄ alkyl. In some embodiments, R¹ is methyl. In some embodiments, R¹ is ethyl. In some embodiments, R¹ is n-propyl. In some embodiments, R¹ is n-butyl. In some embodiments of a compound of formula II-3, each R¹ is as defined for the compound of formula II-2.

In some embodiments, a compound of formula II-3 is:

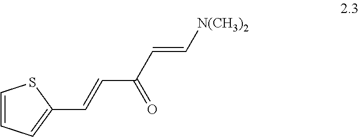 [SMILES: CN(C)/C=C/C(=O)/C=C/c1cccs1]

In some embodiments, compound 2.1 is provided as a solution in toluene. In some embodiments, compound 2.1 is provided from Step 2-1 without purification and/or isolation (e.g., as a solution in toluene).

In some embodiments, suitable conditions in Step 2-2 comprise heating a mixture comprising compound 2.1 and a compound of formula II-2 at a suitable temperature. In some embodiments, Step 2-2 comprises heating a mixture to reflux temperature. In some embodiments, Step 2-2 comprises heating the mixture to between about 100° C. and about 120° C., e.g., about 110° C. In some embodiments, suitable conditions in Step 2-2 comprise heating a mixture comprising compound 2.1 and a compound of formula II-2 for a suitable period of time (e.g., about 12 h, about 24 h, or about 36 h).

In some embodiments, Step 2-2 is performed in a suitable solvent. In some such embodiments, a suitable solvent is an organic solvent. In some embodiments, a suitable solvent is a non-polar aromatic solvent (e.g., toluene or benzene). In some embodiments, a suitable solvent is toluene.

In some embodiments, Step 2-2 further comprises allowing the reaction mixture to cool to, e.g., about 20° C., (e.g., after heating at a suitable temperature for a suitable period of time). In some embodiments, Step 2-2 further comprises concentrating the reaction mixture (e.g., after allowing it to cool). In some embodiments, Step 2-2 further comprises adding ethyl acetate to the concentrated reaction mixture. In some embodiments, Step 2-2 further comprises isolating a compound of formula II-3 via filtration.

### Step 2-3 of Scheme 2

Step 2-3 of Scheme 2 comprises a condensation-cyclization reaction between a compound of formula II-3 and hydrazine to provide terevalefim.

In some embodiments, the present disclosure provides a method comprising steps of:
(i) providing a compound of formula II-3:

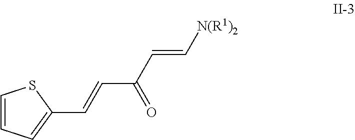

wherein each R¹ is independently C₁₋₆ aliphatic; and
(ii) contacting the compound of formula II-3 with hydrazine under suitable conditions to provide terevalefim.

In some embodiments of a compound of formula II-3, each R¹ is independently C₁₋₆ aliphatic. In some embodiments, each R¹ is independently C₁₋₆ alkyl. In some embodiments, each R¹ is independently C₁₋₄ alkyl. In some embodiments, R¹ is methyl. In some embodiments, R¹ is ethyl. In some embodiments, R¹ is n-propyl. In some embodiments, R¹ is n-butyl.

In some embodiments, a compound of formula II-3 is:

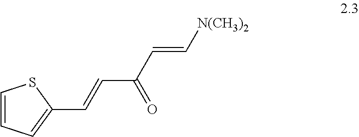 [SMILES: CN(C)/C=C/C(=O)/C=C/c1cccs1]

In some embodiments, Step 2-3 comprises contacting a compound of formula II-3 with hydrazine hydrate. In some embodiments, Step 2-3 comprises contacting a compound of formula II-3 with between 1.0 and 3.0 molar equivalents of hydrazine hydrate, relative to the compound of formula II-3. In some embodiments, Step 2-3 comprises contacting a compound of formula II-3 with between 1.0 and 1.6 molar equivalents of hydrazine hydrate, relative to the compound of formula II-3. In some embodiments, Step 2-3 comprises contacting a compound of formula II-3 with about 1.15 molar equivalents of hydrazine hydrate, relative to the compound of formula II-3. In some embodiments, Step 2-3 comprises contacting a compound of formula II-3 with between 0.5 and 3.0 molar equivalents of hydrazine hydrate, relative to the compound of formula II-3.

In some embodiments, suitable conditions for Step 2-3 comprise a suitable acid. In some such embodiments, a suitable acid is an organic acid. In some embodiments, a suitable acid is a carboxylic acid. In some embodiments, a suitable acid has a pKa between 2.0 and 6.0, e.g., between 4.0 and 5.0. In some embodiments, a suitable acid is acetic acid.

In some embodiments, suitable conditions for Step 2-3 comprise a suitable solvent. In some such embodiments, a suitable solvent is a polar protic solvent. In some embodiments, a suitable solvent is an alcoholic solvent. In some embodiments, a suitable solvent is isopropanol. In some embodiments, a suitable solvent is ethanol.

In some embodiments, suitable conditions for Step 2-3 comprise cooling a mixture of a compound of formula II-3 and a suitable solvent to a particular temperature, e.g., between about 10° C. and about 20° C., e.g., about 15° C. In some embodiments, Step 2-3 comprises adding a suitable acid to the mixture of a compound of formula II-3 and a suitable solvent. In some embodiments, Step 2-3 comprises adding hydrazine to the mixture comprising a compound of formula II-3, a suitable acid, and a suitable solvent, e.g., while maintaining a temperature of from about 10° C. to about 25° C. Alternatively, in some embodiments, Step 2-3 comprises combining a compound of formula II-3 and hydrazine in a suitable solvent, and adding a suitable acid to the resulting mixture, e.g., while maintaining a temperature of from about 10° C. to about 30° C. In some embodiments, Step 2-3 comprises allowing the reaction mixture to warm to a particular temperature between about 15° C. and about 25° C., e.g., about 20° C. (e.g., after combining all components). In some embodiments, Step 2-3 further comprises causing terevalefim to precipitate from the reaction mixture, e.g., by adding water to the reaction mixture. In some embodiments, Step 2-3 further comprises collecting solid terevalefim via filtration.

Without wishing to be bound by theory, a process comprising Steps 2-2 and 2-3 of Scheme 2 can provide certain advantages over a process like that depicted in Scheme 1, e.g., despite an increase in the number of total steps by one. For example, the reagent used in Step 1-2 of Scheme 1 (diethoxyphosphorylacetaldehyde tosylhydrazone) generates byproducts that are difficult to remove and that reduce overall atom economy of the process. In comparison, a process such as that shown in Scheme 2 produces low molecular weight byproducts that are easy to remove. Further, the Scheme 2 process allows for minimization of solvent volume, due to the atom efficiency of reagents used.

### Purification

In some embodiments, the present disclosure provides methods of purifying a composition comprising terevalefim, such as a composition comprising terevalefim obtained from Step 2-3. In some embodiments, purification of such a composition is useful for providing terevalefim in a form suitable for incorporation into a pharmaceutical formulation. For example, in some embodiments, the present disclosure provides methods of purifying compositions comprising terevalefim to remove certain impurities and/or reduce the amount of certain impurities to acceptable levels. In some embodiments, the present disclosure provides methods of purifying compositions comprising terevalefim to provide compositions substantially free of amorphous terevalefim.

In some embodiments, the present disclosure provides a method comprising steps of (i) providing a composition comprising terevalefim (e.g., a composition obtained from Step 2-3 of Scheme 2); (ii) dissolving the composition in a suitable solvent (e.g., ethyl acetate, acetonitrile, etc.); and (iii) filtering the resulting solution over a suitable filtration agent (e.g., alumina, activated carbon, etc.). In some such embodiments, the filtrate is concentrated (e.g., via distillation). In some such embodiments, a suitable anti-solvent (e.g., heptane, hexane, etc.) is added to the concentrated filtrate to induce crystallization. In some such embodiments, the resulting mixture is filtered to provide crystalline terevalefim (e.g., Form A crystalline terevalefim, as described above), which may optionally be further purified by any suitable means.

In some embodiments, the present disclosure provides a method comprising steps of (i) providing a composition comprising terevalefim (e.g., a composition obtained from Step 2-3 of Scheme 2); and (ii) dissolving the composition in a suitable solvent; and (iii) cooling the solution to induce crystallization. In some embodiments, the method further comprises dissolving the composition in a second suitable solvent (e.g., after recrystallizing from the first suitable solvent). For example, in some embodiments, a method comprises (i) providing a composition comprising terevalefim; (ii) dissolving the composition in a suitable solvent (e.g., acetonitrile); (iii) filtering the first mixture to provide a second composition comprising terevalefim; and, optionally, (iv) dissolving the second composition in a second suitable solvent (e.g., ethyl acetate, toluene, etc.). In some embodiments, provided methods further comprise washing a first or second composition comprising terevalefim with heptanes (e.g., after either the first recrystallization, the second recrystallization, or both). In some embodiments, provided methods of purifying provide crystalline terevalefim (e.g., Form A crystalline terevalefim, as described above).

Without wishing to be bound by any particular theory, methods of preparing terevalefim described herein may provide certain advantages, such as providing terevalefim in a form suitable for incorporation into a pharmaceutical formulation. For example, in some embodiments, provided methods can provide terevalefim substantially free of impurities and/or solvents. In some embodiments, a second slurrying step can provide terevalefim substantially free of acetonitrile.

### Synthetic Intermediates and Compositions

The present disclosure also provides synthetic intermediates and compositions thereof useful in the preparation of HGF/SF mimetics, such as terevalefim.

In some embodiments, the present disclosure provides a compound of formula II-3:

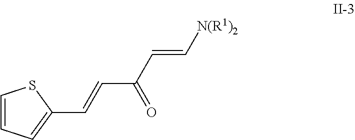

wherein each R¹ is independently C₁₋₆ aliphatic. In some embodiments, each R¹ is independently C₁₋₆ alkyl. In some embodiments, each R¹ is independently C₁₋₄ alkyl. In some embodiments, R¹ is methyl. In some embodiments, R¹ is ethyl. In some embodiments, R¹ is n-propyl. In some embodiments, R¹ is n-butyl.

In some embodiments, a compound of formula II-3 is:

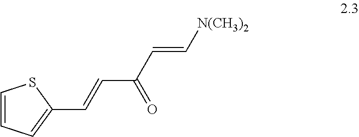 [SMILES: CN(C)/C=C/C(=O)/C=C/c1cccs1]

In some embodiments, the present disclosure provides a composition comprising (i) compound 1.1:

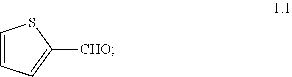

and
(ii) compound 2.1:

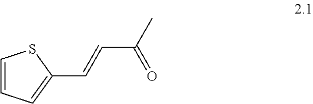

In some such embodiments, a composition comprises (i) compound 1.1; (ii) compound 2.1; and (iii) acetone.

In some embodiments, the present disclosure provides a composition comprising (i) compound 2.1:

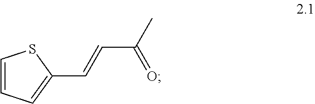

and
(ii) a compound of formula II-3:

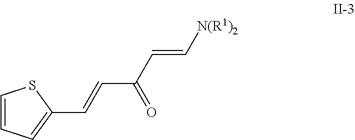

wherein R¹ is as defined above and described in classes and subclasses herein. In some such embodiments, a composition comprises (i) compound 2.1; (ii) a compound of formula II-3; and
(iii) a compound of formula II-2:

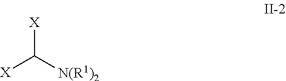

wherein X and R¹ are as defined above and described in classes and subclasses herein. In some embodiments, a composition comprises (i) compound 2.1; (ii) a compound of formula II-3; (iii) a compound of formula II-2; and (iv) toluene.

In some embodiments, the present disclosure provides a composition comprising (i) compound 2.1:

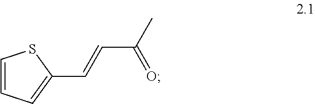

and
(ii) a compound of formula of formula II-2:

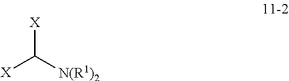

wherein X and R¹ are as defined above and described in classes and subclasses herein. In some such embodiments, a composition comprises (i) compound 2.1; (ii) a compound of formula II-2; and (iii) toluene.

In some embodiments, the present disclosure provides a composition comprising (i) a compound of formula II-3:

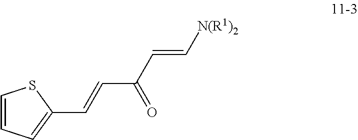

wherein R¹ is as defined above and described in classes and subclasses herein; and (ii) terevalefim. In some such embodiments, a composition comprises (i) a compound of formula II-3; (ii) terevalefim; and (iii) hydrazine. In some such embodiments, a composition comprises (i) a compound of formula II-3; (ii) terevalefim; (iii) hydrazine; and (iv) acetic acid.

In some embodiments, the present disclosure provides a composition comprising (i) a compound of formula II-3:

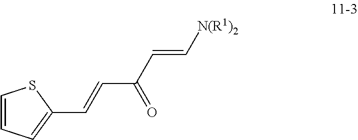

wherein R¹ is as defined above and described in classes and subclasses herein; and (ii) acetic acid. In some such embodiments, a composition comprises (i) a compound of formula II-3; (ii) acetic acid; and (iii) hydrazine. In some embodiments, a composition comprises (i) compound 2.3; and (ii) hydrazine.

In some embodiments, the present disclosure provides compositions comprising terevalefim and/or certain synthetically useful intermediates in the absence of certain impurities and/or in the presence of minimal amounts of certain impurities.

In some embodiments, the present disclosure provides a composition comprising terevalefim that is substantially free of acetonitrile, e.g., obtainable according to methods described herein. In some embodiments, a composition comprises terevalefim and an amount of acetonitrile acceptable for administration to humans. See, e.g., the International Council for Harmonisation of Technical Requirements for Pharmaceuticals for Human Use (ICH)'s Guidelines for Residual Solvents, Q3C(R6), Oct. 20, 2016. In some embodiments, a composition comprises terevalefim and less than 410 ppm acetonitrile. Acetonitrile concentration can be measured using any suitable means known in the art, for example, gas chromatography. For example, in some embodiments, acetonitrile concentration is determined as described in Gas Chromatography Method A of Example 7.

In some embodiments, the present disclosure provides a composition comprising terevalefim and hydrazine. In some embodiments, the present disclosure provides a composition comprising terevalefim that is substantially free of hydrazine, e.g., obtainable according to methods described herein. In some embodiments, a composition comprises terevalefim and an amount of hydrazine acceptable for administration to humans. See, e.g., ICH's Guidelines for Assessment and Control of DNA Reactive (Mutagenic) Impurities in Pharmaceuticals to Limit Potential Carcinogenic Risk, M7(R1), Mar. 31, 2017. In some embodiments, a composition comprises terevalefim and less than 10 ppm hydrazine. Hydrazine concentration can be measured using any suitable means known in the art, for example, high pressure liquid chromatography (HPLC). In some embodiments, hydrazine concentration is determined as described in HPLC Method A of Example 7.

In some embodiments, the present disclosure provides a composition comprising terevalefim and 3-[(1E)-2-(thiophen-3-yl)ethen-1-yl]-1H-pyrazole (3):

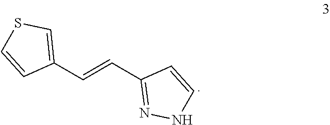 [SMILES: C(=C\c1cc[nH]n1)/c1ccsc1]

In some embodiments, the present disclosure provides a composition comprising terevalefim that is substantially free of compound 3, e.g., obtainable according to methods described herein. In some embodiments, a composition comprises terevalefim and less than about 1.0 area %, less than about 0.5 area %, or less than about 0.3 area % compound 3, as judged by HPLC. In some embodiments, a composition comprises terevalefim and between about 0.05 area % and 1.0 area %, between about 0.05 area % and about 0.5 area %, or between about 0.05 area % and about 0.3 area % compound 3, as judged by HPLC. In some embodiments, the amount of compound 3 is determined as area percent of an HPLC chromatograph obtained as described in HPLC Method B or HPLC Method C of Example 7.

In some embodiments, the present disclosure provides a composition comprising terevalefim and Impurity A. “Impurity A” as used herein describes the compound or compounds with a relative retention time (RRT), i.e., relative to the retention time of terevalefim, of 1.33 when the crude reaction mixture of Example 1 Step 3 is analyzed via HPLC according to HPLC Method B of Example 7. In some embodiments, a composition comprises terevalefim substantially free of Impurity A, e.g., obtainable according to methods described herein. In some embodiments, a composition comprises terevalefim and less than about 0.2 area %, less than about 0.1 area %, or less than about 0.05 area % Impurity A, as judged by HPLC. In some embodiments, a composition comprises terevalefim and between about 0.05 area % and 0.2 area % or between about 0.05 area % and about 0.1 area % Impurity A. In some embodiments, the amount of Impurity A is determined as area percent of an HPLC chromatograph obtained as described in HPLC Method B of Example 7.

### Provided Formulations of Terevalefim

The present disclosure also provides compositions and formulations of HGF/SF mimetics, such as terevalefim. In some embodiments, such compositions and formulations achieve certain desirable results, e.g., in comparison to previously reported HGF/SF mimetic-containing compositions and formulations.

As described above, terevalefim has been demonstrated to be remarkably useful for treatment of a variety of conditions and is or has been the subject of a number of clinical trials, e.g., for delayed graft function in recipients of a deceased donor kidney (Clinicaltrials.gov identifier: NCT02474667), acute kidney injury after cardiac surgery involving cardiopulmonary bypass (Clinicaltrials.gov identifier: NCT02771509), and COVID-19 pneumonia (Clinicaltrials.gov identifier: NCT04459676). In each of these clinical trials, terevalefim has been and/or is administered to subjects as a liquid formulation via an intravenous infusion.

Certain liquid (e.g., for intravenous or intraperitoneal administration) and solid (e.g., for oral administration) formulations of terevalefim have been described. See, for example, PCT Application No. PCT/US2009/004014, filed Jul. 9, 2009 and published as WO2010/005580 on Jan. 14, 2010 (“the '580 application”), the entirety of which is hereby incorporated by reference. In particular, the '580 application generally describes liquid formulations comprising HGF/SF mimetics that have increased solubility in the formulation as compared to in aqueous buffer such as phosphate-buffered saline. The '580 application generally describes liquid compositions comprising HGF/SF mimetics with increased solubility, wherein said compositions comprise polyethylene glycol, polysorbate or a combination thereof. The '580 application generally describes liquid compositions comprising HGF/SF mimetics, wherein said composition comprises polyethylene glycol 300 and/or polysorbate 80. The '580 application generally describes liquid compositions comprising HGF/SF mimetics, wherein said composition comprises about 40% to about 60% (v/v) and/or about 50% (v/v) polyethylene glycol. The '580 application generally describes liquid compositions comprising HGF/SF mimetics, wherein said composition comprises about 5% to about 15% (v/v) and/or about 10% (v/v) polysorbate. The '580 application generally describes liquid compositions comprising HGF/SF mimetics, wherein said composition comprises 50% (v/v) polyethylene glycol together with 10% (v/v) polysorbate 80. The '580 application generally describes liquid compositions comprising HGF/SF mimetics, wherein the balance of the solution can be a saline solution, a buffer or a buffered saline solution, such as phosphate-buffered saline. The '580 application generally describes liquid compositions comprising HGF/SF mimetics, wherein the pH of the solution can be from about pH 5 to about pH 9, and/or from about pH 6 to about pH 8, and/or pH 7.4. The '580 application generally describes liquid compositions comprising HGF/SF mimetics, wherein the HGF/SF mimetic compound is soluble at a concentration higher than in buffer alone, and can be present at about 0.8 to about 10 mg/mL of solution, or even higher. See paragraph [0146] of the '580 application. The '580 application also describes, in paragraph [0279], a specific formulation of terevalefim, wherein the formulation comprises 0.5% (w/v) terevalefim in 10% polysorbate 80 (v/v), 50% polyethylene glycol 300 (v/v) and 40% (v/v) phosphate-buffered saline.

Certain characteristics and/or features of a liquid composition comprising an active agent (such as terevalefim) are desirable. For example, it is preferable for an intravenous formulation to be a solution (e.g., as opposed to a suspension). While some suspensions can be administered intravenously, solutions are less likely to present issues of clogging and/or poor injectability. Additionally, solutions often provide increased uniformity for delivery of an active agent. Yet, a liquid formulation that is a solution must also have a suitable viscosity for efficient intravenous administration. Furthermore, formulations administered intravenously must be aqueous, though additional water-soluble and/or water-miscible excipients may also be present. It is desirable for formulations administered intravenously to have a suitable osmolality, e.g., an osmolality comparable to the natural osmolality of blood. Additional constraints include the need to deliver a suitable quantity of an active agent in a reasonable overall volume of fluid. Formulations for intravenous administration must therefore be optimized to balance the solubility of an active agent and a desire to minimize unnecessary fluid administration. In certain patient populations, minimization of fluid administration is particularly important. For example, in patients with poor kidney function, it is desirable to avoid administration of unnecessary fluids, in order to minimize the amount of fluid their kidneys need to process. Moreover, any formulation for intravenous administration must be stable to storage and/or be provided in a format that is stable to storage and is easily modified to provide the administered formulation. For example, it may be desirable to initially provide a more concentrated formulation for ease of manufacturing and/or shipping and/or storage, which is then diluted just prior to administration by a medical professional.

In particular, formulation of terevalefim as a liquid composition for intravenous administration presented several challenges. First, terevalefim is poorly soluble in water. Terevalefim has an intrinsic solubility of 2.17 mg/mL in water at pH<2 (e.g., when measured by HPLC). Therefore, additional excipients were required to prepare a liquid formulation of terevalefim that was a solution, could be manufactured and/or shipped and/or stored in a reasonable volume, and could be administered as a reasonable volume of fluid (e.g., less than 50 mL or less than 100 mL per dose). Preparing a formulation of terevalefim with a suitable osmolality also presented a challenge. Without wishing to be bound by theory, the use of additional excipients in order to solubilize terevalefim may be a source of increased osmolality observed in certain formulations. The present disclosure provides liquid formulations of terevalefim, as well as improvements upon various formulations generally described in the '580 application.

In some embodiments, the present disclosure provides liquid formulations of terevalefim. In some embodiments, provided formulations comprise from about 0.8 mg/mL to about 10 mg/mL terevalefim. In some embodiments, provided formulations comprise from about 6 mg/mL to about 100 mg/mL terevalefim, from about 6 mg/mL to about 80 mg/mL, from about 6 mg/mL to about 60 mg/mL, from about 6 mg/mL to about 40 mg/mL, from about 6 mg/mL to about 20 mg/mL, from about 10 mg/mL to about 100 mg/mL, from about 10 mg/mL to about 80 mg/mL, from about 10 mg/mL to about 60 mg/mL, from about 10 mg/mL to about 40 mg/mL, or from about 10 mg/mL to about 20 mg/mL In some embodiments, provided formulations comprise from about 6 mg/mL to about 10 mg/mL terevalefim. In some embodiments, provided formulations comprise about 6 mg/mL terevalefim. In some embodiments, provided formulations comprise about 10 mg/mL terevalefim.

In some embodiments, provided formulations of terevalefim comprise polyethylene glycol (e.g., polyethylene glycol 300). In some embodiments, provided formulations comprise from about 10% (w/v) to about 90% (w/v), from about 10% (w/v) to about 65% (w/v), from about 20% (w/v) to about 80% (w/v), from about 20% (w/v) to about 60/(w/v), from about 30% (w/v) to about 70% (w/v), or from about 40% (w/v) to about 60% (w/v) polyethylene glycol (e.g., polyethylene glycol 300). In some embodiments, provided formulations comprise about 10% (w/v), about 20% (w/v), about 30% (w/v), about 40% (w/v), about 50% (w/v), about 60% (w/v), about 70% (w/v), about 80% (w/v), or about 90% (w/v) polyethylene glycol (e.g., polyethylene glycol 300). In some embodiments, provided formulations comprise about 30% (w/v) polyethylene glycol (e.g., polyethylene glycol 300). In some embodiments, provided formulations comprise about 50% (w/v) polyethylene glycol (e.g., polyethylene glycol 300).

In some embodiments, provided formulations of terevalefim comprise polysorbate (e.g., polysorbate 80). In some embodiments, provided formulations comprise from about 1% (w/v) to about 25% (w/v), from about 2% (w/v) to about 20% (w/v), or from about 5% (w/v) to about 15% (w/v) polysorbate (e.g., polysorbate 80). In some embodiments, provided formulations comprise about 1% (w/v), about 2% (w/v), about 3% (w/v), about 4% (w/v), about 5% (w/v), about 6% (w/v), about 10% (w/v), about 15% (w/v), about 20% (w/v), or about 25% (w/v) polysorbate (e.g., polysorbate 80). In some embodiments, provided formulations comprise about 6% (w/v) polysorbate (e.g., polysorbate 80). In some embodiments, provided formulations comprise about 10% (w/v) polysorbate (e.g., polysorbate 80).

In some embodiments, provided formulations of terevalefim are aqueous. In some embodiments, provided formulations comprise aqueous components, such as aqueous buffer, normal saline, or buffered saline (e.g., phosphate buffered saline). In some embodiments, provided formulations comprise aqueous components, such as normal saline or buffered saline (e.g., phosphate buffered saline) or a combination of both. In some embodiments, aqueous buffer is any suitable aqueous buffer. In some embodiments, aqueous buffer is an aqueous phosphate buffer, i.e., an aqueous solution comprising one or more phosphate salts (e.g., monobasic potassium phosphate and/or dibasic sodium phosphate). In some embodiments, aqueous buffer comprises monobasic potassium phosphate and dibasic sodium phosphate in a weight ratio of 1.76:7.26. In some embodiments, aqueous buffer is an aqueous acetate buffer, i.e., an aqueous solution comprising one or more acetate salts. In some embodiments, aqueous buffer is an aqueous citrate buffer, i.e., an aqueous solution comprising one or more citrate salts. In some embodiments, phosphate-buffered saline is an aqueous solution comprising one or more phosphate salts (e.g., monobasic potassium phosphate and/or dibasic sodium phosphate) and one or more chloride salts (e.g., sodium chloride and/or potassium chloride). In some embodiments, phosphate-buffered saline comprises 0.2 mg/mL potassium chloride, 0.2 mg/mL monobasic potassium phosphate, 8.0 mg/mL sodium chloride, and 2.16 mg/mL sodium phosphate dibasic heptahydrate. In some embodiments, normal saline is an aqueous solution comprising one or more chloride salts (e.g., sodium chloride and/or potassium chloride). In some embodiments, normal saline comprises 0.9% sodium chloride by weight.

In some embodiments, provided formulations comprise aqueous components in an amount suitable to balance other components (e.g., to bring the total volume to 100% of the desired volume). In some embodiments, provided formulations comprise from about 100% (w/v) to about 90% (w/v), from about 20% (w/v) to about 80% (w/v), from about 30% (w/v) to about 70% (w/v), or from about 40% (w/v) to about 60% (w/v) aqueous components. In some embodiments, provided formulations comprise about 10% (w/v), about 20% (w/v), about 30% (w/v), about 40% (w/v), about 50% (w/v), about 60% (w/v), about 70% (w/v), about 80% (w/v), or about 90% (w/v) aqueous components. In some embodiments, provided formulations comprise about 40% (w/v) aqueous components (e.g., phosphate buffered saline). In some embodiments, provided formulations comprise about 40% (w/v) aqueous components (e.g., aqueous buffer, e.g., aqueous phosphate buffer). In some embodiments, provided formulations comprise about 64% (w/v) aqueous components (e.g., normal saline and phosphate buffered saline). In some embodiments, provided formulations comprise about 64% (w/v) aqueous components (e.g., aqueous buffer, e.g., aqueous phosphate buffer). In some embodiments, provided formulations comprise about 24% (w/v) phosphate buffered saline and about 40% (w/v) normal saline.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80); and
one or more aqueous components.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80); and aqueous buffer.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and
one or more aqueous components.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and aqueous buffer.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and
one or more aqueous components selected from phosphate buffered saline and normal saline.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and aqueous buffer.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and
about 40% (w/v) phosphate buffered saline.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and aqueous buffer.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80); and
one or more aqueous components.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80); and aqueous buffer.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

In some embodiments, the present disclosure provides a formulation comprising:
about 6 mg/mL terevalefim;
about 30/c (w/v) polyethylene glycol (e.g., polyethylene glycol 300);
about 6% (w/v) polysorbate (e.g., polysorbate 80); and
about 64% (w/v) aqueous buffer.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
about 24% (w/v) phosphate buffered saline; and
about 40% (w/v) normal saline.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80);
one or more aqueous components (e.g., aqueous buffer); and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0/%, or between about 0.05% and about 0.5%, or between about 0.05% and about 0.3%.

In some embodiments, the present disclosure provides a formulation comprising:
about 10 mg/mL terevalefim;
about 50% (w/v) polyethylene glycol (e.g., polyethylene glycol 300);
about 10/c (w/v) polysorbate (e.g., polysorbate 80); and
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 10 mg/mL terevalefim;
about 50% (w/v) polyethylene glycol (e.g., polyethylene glycol 300);
about 100/c (w/v) polysorbate (e.g., polysorbate 80); and
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80);
about 40% (w/v) phosphate buffered saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components (e.g., aqueous buffer); and compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 200/c (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components (e.g., aqueous buffer); and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
about 64% (w/v) aqueous buffer; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
about 24% (w/v) phosphate buffered saline;
about 40% (w/v) normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80);
one or more aqueous components (e.g., aqueous buffer); and
about 0.003 mg/mL to about 0.10 mg/mL, about 0.003 mg/mL to about 0.05 mg/mL, or about 0.003 mg/mL to about 0.03 mg/mL compound 3.

In some embodiments, the present disclosure provides a formulation comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.003 mg/mL to about 0.10 mg/mL, about 0.003 mg/mL to about 0.05 mg/mL, or about 0.003 mg/mL to about 0.03 mg/mL compound 3.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and
one or more aqueous components; and
about 0.005 mg/mL to about 0.10 mg/mL, about 0.005 mg/mL to about 0.05 mg/mL, or about 0.005 mg/mL to about 0.03 mg/mL compound 3.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80); and
one or more aqueous components selected from phosphate buffered saline and normal saline; and
about 0.005 mg/mL to about 0.10 mg/mL, about 0.005 mg/mL to about 0.05 mg/mL, or about 0.005 mg/mL to about 0.03 mg/mL compound 3.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components; and
about 0.005 mg/mL to about 0.10 mg/mL, about 0.005 mg/mL to about 0.05 mg/mL, or about 0.005 mg/mL to about 0.03 mg/mL compound 3.

In some embodiments, the present disclosure provides a formulation comprising:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.005 mg/mL to about 0.10 mg/mL, about 0.005 mg/mL to about 0.05 mg/mL, or about 0.005 mg/mL to about 0.03 mg/mL compound 3.

In some embodiments, the present disclosure provides a formulation comprising:
about 10 mg/mL terevalefim;
about 50% (w/v) polyethylene glycol (e.g., polyethylene glycol 300);
about 10% (w/v) polysorbate (e.g., polysorbate 80);
about 40% (w/v) phosphate buffered saline; and
about 0.005 mg/mL to about 0.10 mg/mL, about 0.005 mg/mL to about 0.05 mg/mL, or about 0.005 mg/mL to about 0.03 mg/mL compound 3.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components (e.g., aqueous buffer); and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components (e.g., aqueous buffer); and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a formulation comprising:
about 6 mg/mL terevalefim;
about 30% (w/v) polyethylene glycol (e.g., polyethylene glycol 300);
about 6% (w/v) polysorbate (e.g., polysorbate 80);
about 64% (w/v) aqueous buffer; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a formulation comprising:
about 6 mg/mL terevalefim;
about 30/c (w/v) polyethylene glycol (e.g., polyethylene glycol 300);
about 6% (w/v) polysorbate (e.g., polysorbate 80);
about 24% (w/v) phosphate buffered saline;
about 40% (w/v) normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides liquid formulations of terevalefim suitable for storage for a particular period of time. For example, in some embodiments, provided formulations can be and/or are stored for at least 1 day, 2 days, 3 days, 1 week, 2 weeks, 1 month, 2 months, 3 months, 6 months, 1 year, 2 years, or 3 years, or longer.

In some embodiments, provided formulations are stored at a particular temperature. For example, in some embodiments, provided formulations are stored at or below 8° C. In some embodiments, provided formulations are stored at about −70° C., about −20° C., about 5° C., or about 30° C. In some embodiments, provided formulations are stored between about 2° C. and about 8° C.

In some embodiments, provided formulations are stored at a particular temperature for a particular period of time. For example, in some embodiments, provided formulations are stored at about −20° C., about 5° C., or about 30° C. for a particular period of time (e.g., 1 day, 2 days, 3 days, 1 week, 2 weeks, 1 month, 2 months, 3 months, 6 months, 1 year, 2 years, or 3 years, or longer). In some embodiments, provided formulations are stored at about −70° C. for a particular period of time (e.g., 1 day, 2 days, 3 days, 1 week, 2 weeks, 1 month, 2 months, 3 months, 6 months, 1 year, 2 years, or 3 years, or longer). In some embodiments, provided formulations are stored at or below 8° C. for a particular period of time (e.g., 1 day, 2 days, 3 days, 1 week, 2 weeks, 1 month, 2 months, 3 months, 6 months, 1 year, 2 years, or 3 years, or longer). In some embodiments, provided formulations are stored between about 2° C. and about 8° C. for a particular period of time (e.g., 1 day, 2 days, 3 days, 1 week, 2 weeks, 1 month, 2 months, 3 months, 6 months, 1 year, 2 years, or 3 years, or longer).

In some embodiments, provided formulations are stored at a particular temperature for no more than a particular period of time. For example, in some embodiments, provided formulations are stored between about 20° C. and about 25° C. for no more than one month. In some embodiments, provided formulations are stored between about 15° C. and about 30° C. for no more than one month.

In some embodiments, provided formulations are stored under an inert atmosphere, e.g., under nitrogen or argon.

Without wishing to be bound by theory, the present disclosure encompasses the recognition that certain formulations of terevalefim can be stored for a longer period of time when the pH of the formulation is maintained within a certain range of values. As used herein, the pH of a formulation is the pH value obtained when measured with a pH meter without any modification or dilution of the formulation. It will be appreciated that although “pH” often refers to the pH of aqueous solutions, throughout this disclosure, “pH” is also used to describe the pH of provided formulations (which may not be completely aqueous) when measured with a pH meter without any modification or dilution of the formulation. Accordingly, in some embodiments, provided compositions have a pH from about 5.0 to about 9.0, from about 6.0 to about 8.0, from about 6.4 to about 8.4, or from about 7.4 to about 7.9. In some embodiments, provided compositions have a pH of about 7.4. In some embodiments, provided compositions have a pH of from about 5.0 to about 9.0, from about 6.0 to about 8.0, from about 6.4 to about 8.4, or from about 7.4 to about 7.9 after being stored at a particular temperature (e.g., −20° C., about 5° C., or about 30° C.) for a particular period of time (e.g., 1 day, 2 days, 3 days, 1 week, 2 weeks, 1 month, 2 months, 3 months, 6 months, 1 year, 2 years, or 3 years, or longer). In some embodiments, provided compositions have a pH of from about 5.0 to about 9.0, from about 6.0 to about 8.0, from about 6.4 to about 8.4, or from about 7.4 to about 7.9 after being stored at or below 8° C. for a particular period of time (e.g., 1 day, 2 days, 3 days, 1 week, 2 weeks, 1 month, 2 months, 3 months, 6 months, 1 year, 2 years, or 3 years, or longer).

In some embodiments, the present disclosure encompasses the recognition that the amount of aqueous buffer and/or buffered saline (e.g., phosphate buffered saline) relative to one or more other components (e.g., terevalefim) can affect the pH of provided formulations, particularly as they are stored over time. For example, as described in Example 5 below, Formulation A (comprising 10 mg/mL terevalefim and 40% (w/v) phosphate buffered saline) demonstrated little change in pH over 60 months when stored at −20° C. or 5° C., while Formulation B (comprising 6 mg/mL terevalefim and 24% (w/v) phosphate buffered saline) displayed a decrease in pH from 6.7 to 6.1 over 6 months when stored at 5° C. Without wishing to be bound by theory, it will be appreciated that the pH of formulations comprising relatively less buffered saline (e.g., phosphate buffered saline) may decrease over time, possibly resulting in degradation of terevalefim. Accordingly, in some embodiments, provided formulations comprise at least about 30% (w/v), about 40% (w/v), or about 50% (w/v) aqueous buffer (e.g., aqueous phosphate buffer) or buffered saline (e.g., phosphate-buffered saline). In some embodiments, provided formulations comprising 10 mg/mL terevalefim comprise at least about 30% (w/v), about 40% (w/v), or about 50% (w/v) buffered saline (e.g., phosphate-buffered saline). In some embodiments, provided formulations comprising 10 mg/mL terevalefim comprise at least about 30% (w/v), about 40% (w/v), or about 50% (w/v) aqueous buffer (e.g., aqueous phosphate buffer). In some embodiments, provided formulations comprising 6 mg/mL terevalefim comprise at least about 30% (w/v), about 40% (w/v), or about 50% (w/v) buffered saline (e.g., phosphate-buffered saline). In some embodiments, provided formulations comprising 6 mg/mL terevalefim comprise at least about 30% (w/v), about 40% (w/v), about 50% (w/v), about 60% (w/v), about 65% (w/v), or about 70% (w/v) aqueous buffer (e.g., aqueous phosphate buffer).

Without wishing to be bound by theory, the present disclosure encompasses the recognition that certain formulations of terevalefim may be stored for a longer period of time when the buffer capacity of the formulation is maintained within a certain range of values. As used herein, “buffer capacity” refers to a magnitude of resistance of a buffer solution to a change in pH upon addition of an acid or base. In some embodiments, buffer capacity is expressed as a ratio of the change in added acid or base to the change in pH. In some embodiments, buffer capacity is measured by providing a formulation of terevalefim with a pH of about 8.5, titrating the formulation until the pH is about 5.0, and calculating the amount of acid required to effect the change in pH. In some embodiments, a provided formulation has a buffer capacity comparable to that of a reference formulation (e.g., a formulation that has been demonstrated to maintain a pH within a particular range after being stored at a particular temperature for a particular period of time). In some embodiments, a reference formulation has been demonstrated to maintain a pH from about 5.0 to about 9.0, from about 6.0 to about 8.0, from about 6.4 to about 8.4, or from about 7.4 to about 7.9 after being stored at about −20° C., about 5° C., or about 30° C. for a particular period of time (e.g., 1 day, 2 days, 3 days, 1 week, 2 weeks, 1 month, 2 months, 3 months, 6 months, 1 year, 2 years, or 3 years, or longer).

In some embodiments, the present disclosure encompasses the recognition that the buffer capacity of a provided formulation can affect pH of provided formulations, particularly as they are stored over time. Without wishing to be bound by theory, it will be appreciated that the formulations comprising relatively less aqueous buffer and/or buffered saline (e.g., phosphate buffered saline) may have a lower buffer capacity, possibly resulting in degradation of terevalefim, e.g., as it is stored overtime. Accordingly, in some embodiments, provided formulations comprise at least about 30% (w/v), about 40% (w/v), or about 50% (w/v) aqueous buffer or buffered saline (e.g., phosphate-buffered saline). In some embodiments, provided formulations comprising 10 mg/mL terevalefim comprise at least about 30% (w/v), about 40% (w/v), or about 50% (w/v) buffered saline (e.g., phosphate-buffered saline). In some embodiments, provided formulations comprising 10 mg/mL terevalefim comprise at least about 30% (w/v), about 40% (w/v), or about 50% (w/v) aqueous buffer. In some embodiments, provided formulations comprising 6 mg/mL terevalefim comprise at least about 30% (w/v), about 40% (w/v), or about 50% (w/v) buffered saline (e.g., phosphate-buffered saline). In some embodiments, provided formulations comprising 6 mg/mL terevalefim comprise at least about 30% (w/v), about 40% (w/v), about 50% (w/v), or about 600 (w/v) aqueous buffer.

As noted above, the present disclosure encompasses the recognition that, in some embodiments, when the pH of provided formulations drops below a certain value (e.g., below about 8.0, about 7.5, about 7.0, or about 6.5), degradation products of terevalefim are observed. For example, in some embodiments, provided compositions (e.g., those that have a pH below about 8.0, about 7.5, about 7.0, or about 6.5 and/or that have been stored for about 3 months, about 6 months, about 1 year, about 2 years, about 3 years, or longer, e.g., at a particular temperature such as −20° C., 5° C., or 25° C.) comprise small amounts (e.g., from about 0.1 area % to about 0.6 area % as judged by HPLC Method B or HPLC Method C of Example 7) of compound 1.1:

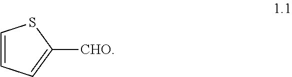

Accordingly, in some embodiments, the present disclosure encompasses the recognition that adjusting the pH and/or increasing the relative amount of buffered saline (e.g., phosphate buffered saline) in provided compositions can eliminate and/or reduce degradation of terevalefim to degradants such as compound 1.1. In some embodiments, the present disclosure encompasses the recognition increasing the buffer capacity and/or increasing the relative amount of aqueous buffer or buffered saline (e.g., phosphate buffered saline) in provided compositions can eliminate and/or reduce degradation of terevalefim into degradants such as compound 1.1.

In some embodiments, provided formulations comprise compound 1.1 in an amount below a particular threshold value. In some embodiments, provided formulations comprise compound 1.1 in an amount below a particular threshold value after having been stored for a particular period time, e.g., as described herein. In some embodiments, provided formulations comprise less than about 1.0 area %, less than about 0.6 area %, less than about 0.4 area %, less than about 0.1 area %, or less than about 0.05 area % as judged by HPLC (e.g., HPLC Method B or HPLC Method C of Example 7). In some embodiments, provided formulations comprise between about 0.05 area % and about 1.0 area %, between about 0.05 area % and about 0.6 area %, between about 0.05 area % and about 0.4 area %, or between about 0.05 area % and about 0.1 area % as judged by HPLC (e.g., HPLC Method B or HPLC Method C of Example 7).

The present disclosure also encompasses the recognition that the color of provided formulations may be indicative of a decreased pH and/or increase in degradation of terevalefim, e.g., when provided formulations are stored for a particular period of time (e.g., as described herein) and/or at a particular temperature (e.g., as described herein). For example, in some embodiments, provided formulations are colorless, light yellow, or amber. In some embodiments, provided formulations turn from colorless to light yellow to amber after being stored for a particular period of time (e.g., as described herein) and/or at a particular temperature (e.g., as described herein). Changes in color can be determined qualitatively by sight and/or semi-quantitatively using standardized color scales, such as the Lovibond® color comparator with associated Ph. Eur color wheels. In some embodiments, a change in color of a provided formulation correlates with a change in pH of a formulation and/or an increase in degradants (e.g., a darkening in color of the formulation correlates with a decrease in pH and/or an increase in degradants).

In some embodiments, the present disclosure proposes that the source of one or more less desirable features described herein (e.g., decreased pH, increased degradants, and/or darkened color) may be attributable to an excipient of provided formulations. In some embodiments, the present disclosure proposes that polysorbate (e.g., polysorbate 80, e.g., in the amount utilized in certain formulations described herein) may be the source of decreased pH, increased degradants and/or darkened color, particularly in formulations that have been stored for a particular period of time (e.g., as described herein) and/or at a particular temperature (e.g., as described herein). Accordingly, in some embodiments, the present disclosure provides formulations comprising less than a threshold quantity of polysorbate (e.g., polysorbate 80). In some embodiments, provided formulations comprise no polysorbate (e.g., polysorbate 80). In some embodiments, provided formulations comprise less than about 10% (w/v), less than about 6% (w/v), less than about 5% (w/v), less than about 4% (w/v), less than about 3% (w/v), less than about 2% (w/v), or less than about 1% (w/v) polysorbate (e.g., polysorbate 80).

In some embodiments, the present disclosure encompasses the recognition that provided formulations which have a suitable osmolality may be better suited for administration to humans. For example, formulations for intravenous administration to humans typically have an osmolality of less than about 900 mOsm/kg. Osmolality of provided formulations can be estimated or measured using any means suitable for solutions comprising non-aqueous components, such as polyethylene glycol and/or polysorbate. In some embodiments, osmolality may be estimated using the sodium chloride equivalents method or with the equation: Osmolality=Σᵢηᵢ ϕᵢCᵢ, where η=number of particles, ϕ=the osmolar constant, and C=concentration (molality). In some embodiments, the present disclosure encompasses the recognition that use of aqueous buffer instead of, e.g., normal saline and/or phosphate buffered saline, may be beneficial for achieving a desirable osmolality in provided formulations.

In some embodiments, the present disclosure encompasses the recognition that provided formulations which are more concentrated may be better suited for convenient shipping and/or storage, at least because such formulations have less volume and therefore take up less physical space. The inventors surprisingly found that certain formulations of terevalefim in higher concentration are in fact more stable compared to those in lower concentration. For example, a formulation comprising 10 mg/mL terevalefim exhibits less degradation products and color change over time compared to a diluted version of the same formulation (a formulation diluted from 10 mg/mL to 6 mg/mL with normal saline).

As noted above, it is also desirable for provided formulations to be suitable for administration in a reasonable volume. Certain patient populations may benefit from minimization of fluid administration, due to, e.g., poor kidney function. Accordingly, it is desirable to administer provided formulations to patients in minimal volume. In some embodiments, the present disclosure provides formulations that can be administered in a total of less than 60 mL per dose, less than 50 mL per dose, or less than 40 mL per dose.

The present disclosure also encompasses the recognition that optimization of packaging for provided formulations (e.g., vial size) provides certain benefits. In some instances, provided formulations are administered in a weight-based dosing regimen, wherein patients of different weights receive different overall volumes of a particular formulation. It is therefore desirable to ship and/or store provided formulations in packaging that minimizes waste of packaging material and/or minimizes waste of unused formulation and/or reduces risk of contamination from repeated handling of a container. Accordingly, in some embodiments, provided formulations are packaged so that only one container per patient is needed to administer an appropriate dose of terevalefim in most situations. In some embodiments, terevalefim is administered once daily, and therefore only one container is needed per patient per day in order to administer an appropriate dose of terevalefim. In some embodiments, a container comprises from about 20 mL to about 26 mL of a provided formulation. In some embodiments, a container comprises about 23 mL of a provided formulation. In some embodiments, the present disclosure provides about 20 mL to about 26 mL of a provided formulation housed in a vial (e.g., a glass vial). In some embodiments, the present disclosure provides about 23 mL of a provided formulation housed in a vial (e.g., a glass vial).

The present disclosure also provides an article of manufacture comprising a provided formulation (e.g., as described herein) comprising terevalefim; and packaging material comprising a label which indicates that the formulation can be used for particular indications. In some embodiments, the present disclosure provides an article of manufacture comprising from about 20 mL to about 26 mL of a provided formulation (e.g., as described herein) housed in a vial (e.g., a glass vial); and a label which indicates that the formulation can be used for particular indications. In some embodiments, the present disclosure provides an article of manufacture comprising about 23 mL of a provided formulation (e.g., as described herein) housed in a vial (e.g., a glass vial); and a label which indicates that the formulation can be used for particular indications.

### Methods of Preparing Provided Formulations

In some embodiments, the present disclosure provides methods for formulating compounds useful as HGF/SF mimetics, such as terevalefim. As noted above, the '580 application describes preparation of certain liquid formulations of HGF/SF mimetics. In some embodiments, the present disclosure provides certain improvements to methods described in the '580 application. Additionally, the present disclosure, inter alia, encompasses the recognition of certain challenges associated with formulating HGF/SF mimetics such as terevalefim, and in particular, for formulating terevalefim on an industrially relevant scale (e.g., on a scale to produce greater than 380 kg of formulation).

In some embodiments, the present disclosure provides a method comprising steps of: (i) providing terevalefim; (ii) combining terevalefim with suitable excipients to give a mixture; and (iii) adding one or more aqueous components to the mixture, thereby giving a provided formulation as described herein.

In some embodiments, the present disclosure provides a method comprising steps of: (i) providing terevalefim; (ii) combining terevalefim with polysorbate (e.g., polysorbate 80) and polyethylene glycol (e.g., polyethylene glycol 300) to give a mixture; and (iii) adding one or more aqueous components (e.g., phosphate buffered saline, normal saline, or both) to the mixture, thereby giving a provided formulation as described herein.

In some embodiments, the present disclosure provides a method comprising steps of: (i) providing terevalefim; (ii) combining terevalefim with polysorbate (e.g., polysorbate 80) and polyethylene glycol (e.g., polyethylene glycol 300) to give a mixture; and (iii) adding phosphate buffered saline to the mixture, thereby giving a provided formulation as described herein.

In some embodiments, the present disclosure provides a method comprising steps of (i) providing terevalefim; (ii) combining terevalefim with polysorbate (e.g., polysorbate 80) and polyethylene glycol (e.g., polyethylene glycol 300) to give a mixture; (iii) adding water to the mixture; and (iv) adding inorganic salts (e.g., phosphate salts and/or chloride salts) to the mixture, thereby giving a provided formulation as described herein. In some embodiments, provided methods comprise adding phosphate salts (e.g., monobasic potassium phosphate and/or dibasic sodium phosphate) to the mixture. In some embodiments, provided methods further comprise a step of adjusting the pH.

In some embodiments, terevalefim is provided, e.g., for use in methods described herein, in crystalline form. In some embodiments, terevalefim is provided, e.g., for use in methods described herein, as crystalline Form A (see, e.g., Example 3). In some embodiments, terevalefim is provided, e.g., for use in methods described herein, substantially free of amorphous terevalefim. In some embodiments, a composition comprising terevalefim is provided, e.g., for use in methods described herein, substantially free of impurities and/or water and/or other solvents. In some embodiments, the present disclosure provides a method comprising steps of: (i) providing crystalline terevalefim (e.g., Form A); (ii) combining terevalefim with polysorbate (e.g., polysorbate 80) and polyethylene glycol (e.g., polyethylene glycol 300) to give a mixture; and (iii) adding phosphate buffered saline to the mixture, thereby giving a provided formulation as described herein.

In some embodiments, provided methods comprise combining polyethylene glycol (e.g., polyethylene glycol 300) and polysorbate (e.g., polysorbate 80) prior to adding terevalefim. In some embodiments, provided methods comprise combining terevalefim with polyethylene glycol (e.g., polyethylene glycol 300) prior to adding polysorbate (e.g., polysorbate 80). In some embodiments, provided methods comprise combining terevalefim with polysorbate (e.g., polysorbate 80) prior to adding polyethylene glycol (e.g., polyethylene glycol 300).

In some embodiments, provided methods further comprise stirring the mixture of terevalefim, polyethylene glycol (e.g., polyethylene glycol 300), and polysorbate (e.g., polysorbate 80) for a period of time (e.g., prior to adding the aqueous components). In some such embodiments, the mixture is stirred for about 30 minutes, about 60 minutes, or about 90 minutes prior to adding the aqueous components.

Without wishing to be bound by theory, the present disclosure encompasses the recognition that combining terevalefim with polyethylene glycol and polysorbate before adding aqueous components can provide certain benefits and/or advantages. For example, under certain conditions, when terevalefim was added to a mixture of polyethylene glycol 300, polysorbate 80, and aqueous buffered saline, a non-homogeneous mixture was sometimes observed, and sonication was required to achieve homogeneity. As described above, it is desirable for liquid formulations to be homogenous. Additionally, a lack of consistency when producing a formulation on scale is undesirable. Accordingly, the present disclosure encompasses the recognition that the poor aqueous solubility of terevalefim is the likely source of problems with inconsistent formulation. Further, the present disclosure provides at least one solution to the problem, wherein terevalefim is first combined with polyethylene glycol and polysorbate, and then one or more aqueous components are added. An example of such a method is described in Example 4, demonstrating production of greater than 350 L of formulation in one batch. In contrast, batches of just 40 L have been observed to require sonication when terevalefim is added last.

In some embodiments, provided methods further comprise a step of determining homogeneity. In some embodiments, homogeneity of the composition is determined after all components have been added (e.g., after combining terevalefim, polyethylene glycol, polysorbate, and one or more aqueous components). Homogeneity can be determined using any method known in the art. In some embodiments, homogeneity is determined qualitatively by eye. In some embodiments, homogeneity is determined by obtaining a plurality of samples (e.g., from different sections of the batch, such as upper, middle, and lower sections); analyzing each sample for certain characteristics (e.g., appearance, color, amount of terevalefim, impurity content, and/or viscosity); and comparing the results from each sample with the other samples to determine if they are comparable.

In some embodiments, provided methods further comprise a step of determining pH. In some embodiments, provided methods comprise determining pH after all components have been added (e.g., after combining terevalefim, polyethylene glycol, polysorbate, and one or more aqueous components). In some embodiments, provided methods comprise determining pH after terevalefim, polyethylene glycol, polysorbate, water, and inorganic salts (e.g., phosphate salts and/or chloride salts) have been added. As noted above, as used herein, pH of a formulation is the pH obtained when measured with a pH meter without any modification or dilution of the formulation.

In some embodiments, provided methods further comprise a step of adjusting pH. In some embodiments, pH of a formulation is adjusted until it is within a particular range of pH values (e.g., from about 7.4 to about 8.0 or from about 7.5 to about 7.9). In some embodiments, adjusting pH comprises slowly adding acid (e.g., 1 N HCl_{(aq)}) and/or base (e.g, 1 N NaOH_{(aq)}) to the formulation until the pH is within a particular range. In some embodiments, provided methods comprise determining pH; adding small amounts of an acid and/or a base; and determining pH again. In some embodiments, this process is repeated until the pH of the formulation is within a particular range.

In some embodiments, provided methods further comprise a step of filtering. In some embodiments, a formulation and/or mixture resulting from a provided method is filtered to remove undissolved particles. In some embodiments, formulations and/or other mixtures are filtered through polish filters (e.g., 0.45 μm and/or 0.22 μm polish filters).

In some embodiments, provided methods further comprise filling a vial with a provided formulation (e.g., prepared according to a method described herein). In some embodiments, vials are filled with from about 20 mL to about 26 mL of provided formulation. In some embodiments, vials are filled with about 23 mL of provided formulation. In some embodiments, vials are filled and capped with elastomeric stoppers under an atmosphere of nitrogen.

In some embodiments, provided methods are suitable for providing sterilized formulations. In some embodiments, provided methods further comprise a step of sterilizing. In some embodiments, provided methods are performed entirely under aseptic conditions, thereby providing a sterilized formulation once complete. In some embodiments, provided formulations are filled into vials under aseptic conditions. In some embodiments, provided formulations are sterilized using an autoclave at, e.g., about 121° C. or about 115° C. The present disclosure encompasses the recognition that sterilization via certain methods may increase degradation of terevalefim, and therefore certain sterilization procedures (e.g., aseptic filling) are preferable to others (e.g., gamma irradiation and/or heat sterilization).

In some embodiments, provided methods comprise assessing one or more characteristics of a provided formulation, e.g., soon after it has been prepared and/or after it has been stored for a period of time. Such assessments can indicate quality of provided formulations, such as if they are suitable for administration to patients. In some embodiments, the present disclosure provides a method comprising steps of: (i) providing a composition comprising terevalefim, optionally further comprising one or more of polyethylene glycol, polysorbate, and aqueous components; (ii) determining one or more parameters; (iii) comparing the one or more determined parameters with a reference value; and (iv) identifying the composition as suitable for administration when the determined characteristic is comparable to the reference value. In some embodiments, a parameter is pH. In some such embodiments, a reference value is a pH of from about 6.4 to about 8.4, from about 7.4 to about 8.0 or from about 7.5 to about 7.9 when measured as described herein. In some embodiments, a parameter is color. In some such embodiments, a reference value is a color that is colorless or light yellow when judged visually. In some such embodiments, a reference value is B5, B4, B3, or B2 on the EP Color Test Scale. In some embodiments, a parameter is amount of a particular impurity (e.g., compound 1.1). In some such embodiments, a reference value is an amount of compound 1.1 less than about 0.05% or less than about 1.0% as judged by HPLC, as described herein.

### Uses of Terevalefim and Provided Formulations

The present disclosure also provides various methods of using HGF/SF mimetics (such as terevalefim) and formulations thereof. As described above, terevalefim has been demonstrated to be remarkably useful for treatment of a variety of conditions including, for example, fibrotic liver disease, ischemia-reperfusion injury, cerebral infarction, ischemic heart disease, renal disease and renal fibrosis, lung fibrosis, damaged and/or ischemic organs, transplants or grafts, and cerebrovascular disease, among others (see, for example, WO 2004/058721, WO 2010/005580, US 2011/0230407, U.S. Pat. No. 7,879,898, and WO 2009/064422, each of which is hereby incorporated by reference.) Exemplary methods of using terevalefim for, e.g., treating delayed graft function after kidney transplantation and acute lung injury, are described in WO 2021/087392 and WO 2021/183774, each of which is hereby incorporated by reference. Terevalefim is or has been the subject of a number of clinical trials, e.g., for delayed graft function in recipients of a deceased donor kidney (Clinicaltrials.gov identifier: NCT02474667), acute kidney injury after cardiac surgery involving cardiopulmonary bypass (Clinicaltrials.gov identifier: NCT02771509), and COVID-19 pneumonia (Clinicaltrials.gov identifier: NCT04459676).

In some embodiments, the present disclosure provides methods of treating (e.g., lessening the severity of, such as by delaying onset and/or reducing degree and/or frequency of one or more features of) a disease or disorder selected from fibrotic liver disease, ischemia-reperfusion injury, cerebral infarction, ischemic heart disease, renal disease and/or renal fibrosis, lung fibrosis, damaged and/or ischemic organs, transplants or grafts, stroke, and cerebrovascular disease, comprising administering terevalefim (e.g., as a formulation provided herein) to a subject in need thereof.

In some embodiments, the present disclosure provides methods of treating a fibrotic liver disease, comprising administering terevalefim (e.g., as a formulation provided herein) to a subject in need thereof. In some embodiments, a fibrotic liver disease is liver fibrosis or cirrhosis associated with hepatitis C, hepatitis B, delta hepatitis, chronic alcoholism, non-alcoholic steatohepatitis, extrahepatic obstructions, cholangiopathies (e.g., primary biliary cirrhosis or sclerosing cholangitis), autoimmune liver disease, or inherited metabolic disorders (Wilson's disease, hemochromatosis, or alpha-1 antitrypsin deficiency).

In some embodiments, the present disclosure provides methods of treating ischemia-reperfusion injury, comprising administering terevalefim (e.g., as a formulation provided herein) to a subject in need thereof. In some embodiments, the present disclosure provides methods of treating ischemia-reperfusion injury of the liver (e.g., after liver transplantation). In some embodiments, the present disclosure provides methods of treating ischemia-reperfusion injury of the kidney (e.g., after kidney transplantation).

In some embodiments, the present disclosure provides methods of treating cerebral infarction (i.e., stroke), comprising administering terevalefim (e.g., as a formulation provided herein) to a subject in need thereof.

In some embodiments, the present disclosure provides methods of treating ischemic heart disease, comprising administering terevalefim (e.g., as a formulation provided herein) to a subject in need thereof. In some embodiments, the present disclosure provides methods of treating chronic heart failure, comprising administering terevalefim (e.g., as a formulation provided herein) to a subject in need thereof.

In some embodiments, the present disclosure provides methods of treating renal disease and/or renal fibrosis, comprising administering terevalefim (e.g., as a formulation provided herein) to a subject in need thereof. In some embodiments, the present disclosure provides methods of treating chronic renal dysfunction. In some embodiments, the present disclosure provides methods of treating acute renal dysfunction. In some embodiments, the present disclosure provides methods of treating acute kidney injury. In some embodiments, the present disclosure provides methods of treating acute kidney injury associated with cardiac surgery (e.g., cardiac surgery involving cardiopulmonary bypass). In some embodiments, the present disclosure provides methods of treating renal disease associated with ischemia, diabetes, cardiovascular disease, or administration of chemotherapy, antibiotics or radiocontrast agents. In some embodiments, the present disclosure provides methods of treating and/or preventing delayed graft function (e.g., in patients who have received a kidney transplantation).

In some embodiments, the present disclosure provides methods of treating a respiratory disease, disorder or condition, comprising administering terevalefim (e.g., as a formulation provided herein) to a subject in need thereof.

In some embodiments, the present disclosure provides methods of treating lung fibrosis, comprising administering terevalefim (e.g., as a formulation provided herein) to a subject in need thereof. In some embodiments, the present disclosure provides methods of treating idiopathic pulmonary fibrosis.

In some embodiments, the present disclosure provides methods of treating acute lung injury, comprising administering terevalefim (e.g., as a formulation provided herein) to a subject in need thereof. In some embodiments, the present disclosure provides methods of treating acute lung injury. In some embodiments, the present disclosure provides methods of treating acute lung injury associated with COVID-19 pneumonia. In some embodiments, the present disclosure provides methods of treating acute respiratory distress syndrome. In some embodiments, the present disclosure provides methods of treating acute lung injury, acute respiratory distress syndrome, pneumonia (e.g., influenza-associated pneumonia or COVID-19-associated pneumonia), pulmonary edema, TGFβ1-induced lung injury, emphysema, chemically-induced (e.g, chlorine gas) lung injury, thermally-induced (e.g., smoke or burn) lung injury, shock-induced lung injury (e.g., lipopolysaccharide-induced shock), ischemic reperfusion lung injury, hemorrhagic shock lung injury, radiation-induced lung injury, blunt trauma to lung, and lung transplantation injury.

In some embodiments, the present disclosure provides methods of treating a chronic obstructive pulmonary disease such as emphysema, secondary effects of tobacco abuse or smoking, chronic bronchitis, asthma, cystic fibrosis, alpha-1 antitrypsin deficiency, bronchiectasis, or some forms of bullous lung diseases, comprising administering terevalefim (e.g., as a formulation provided herein) to a subject in need thereof.

In some embodiments, the present disclosure provides methods of treating demyelinating diseases and traumatic diseases of the central nervous system, such as spinal cord injury, traumatic brain injury, multiple sclerosis, or hereditary neurodegenerative diseases, such as, but not limited to, leukodystrophies including metachromatic leukodystrophy, Refsum's disease, adrenoleukodystrophy, Krabbe's disease, phenylketonuria, Canavan disease, Pelizaeus-Merzbacher disease and Alexander's disease, comprising administering terevalefim (e.g., as a formulation provided herein) to a subject in need thereof.

In some embodiments, the present disclosure provides methods of treating fibrotic diseases of connective tissue, such as, but not limited to, scleroderma, systemic sclerosis, generalized scleroderma, limited scleroderma and post-surgical adhesions, comprising administering terevalefim (e.g., as a formulation provided herein) to a subject in need thereof.

In some embodiments, the present disclosure provides methods of treating muscular dystrophy, comprising administering terevalefim (e.g., as a formulation provided herein) to a subject in need thereof.

In some embodiments, the present disclosure provides methods of treating amyotrophic lateral sclerosis, comprising administering terevalefim (e.g., as a formulation provided herein) to a subject in need thereof.

In some embodiments, the present disclosure provides methods of treating acute injuries (e.g., acute organ injuries, such as acute lung injury, acute liver injury, or acute kidney injury), as well as for treating chronic injuries (e.g., chronic organ injuries, such as chronic lung injury, chronic liver injury, or chronic kidney injury). In some embodiments, provided methods are useful for treating fibrosis associated with an acute injury, such as that incurred from trauma and/or surgery and/or infection (e.g., a viral infection). In some embodiments, provided methods are useful for treating damaged and/or ischemic organs, transplants, or grafts, as well as ischemia/reperfusion injury or post-surgical scarring.

In some embodiments, the present disclosure also provides methods of administering terevalefim (e.g., as a formulation provided herein). As described herein, provided liquid formulations of terevalefim are particularly suited for intravenous administration (e.g., as a bolus or infusion). In some embodiments, provided formulations are administered as an infusion over about 10 min, about 20 min, about 30 min, or about 40 min. In some embodiments, provided formulations are administered in an amount suitable to provide about 0.5 mg/kg, about 1 mg/kg, about 2 mg/kg, about 3 mg/kg, about 4 mg/kg, about 6 mg/kg, or about 8 mg/kg terevalefim. In some embodiments, provided formulations are administered intravenously at an infusion rate suitable to provide about 0.5 mg/kg, about 1 mg/kg, about 2 mg/kg, about 3 mg/kg, about 4 mg/kg, about 6 mg/kg, or about 8 mg/kg terevalefim over about 10 min, about 20 min, about 30 min, or about 40 min. In some embodiments, provided formulations are administered as an infusion over about 30 min in an amount suitable to provide about 2 mg/kg terevalefim.

In some embodiments, methods provided herein comprise periodic administration of terevalefim (e.g., three or four infusions of terevalefim separated by 24 (+2) hours). In some embodiments, methods provided herein comprise administration of one, two, three, four, or five infusions of terevalefim separated by a regular interval. In some embodiments, methods provided herein comprise administration of six, seven, eight, nine, or ten infusions of terevalefim separated by a regular interval. In some such embodiments, a regular interval can be about 12 hours, about 18 hours, about 24 hours, about 30 hours, or about 36 hours.

In some embodiments, provided methods comprise administering terevalefim (e.g., as a formulation provided herein) once daily for three days. In some embodiments, provided methods comprise administering terevalefim (e.g., as a formulation provided herein) at 2 mg/kg once daily for three days. In some embodiments, provided methods comprise administering terevalefim (e.g., as a formulation provided herein) once daily for four days. In some embodiments, provided methods comprise administering terevalefim (e.g., as a formulation provided herein) at 2 mg/kg once daily for four days.

In some embodiments, terevalefim (e.g., a formulation provided herein) is first administered within about 4 hours, about 6 hours, about 12 hours, about 18 hours, about 24 hours, about 30 hours, about 36 hours, or about 48 hours of an initiating event (e.g., randomization, organ transplantation, surgery, or acute injury).

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and aqueous buffer.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80); and
one or more aqueous components.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80); and aqueous buffer.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
aqueous buffer; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising intravenously administering to a subject a formulation comprising:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
aqueous buffer; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising intravenously administering to a subject a formulation comprising.
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80);
aqueous buffer; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising intravenously administering to a subject a formulation comprising:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, the present disclosure provides a method comprising steps of: (i) providing a first formulation of terevalefim; (ii) diluting the first formulation with normal saline to give a second formulation of terevalefim; and (iii) administering the second formulation to a subject in need thereof. In some such embodiments, a first formulation of terevalefim is more concentrated (e.g., 10 mg/mL) than a second formulation of terevalefim (e.g., 6 mg/mL). In some embodiments, provided methods further comprise diluting the first formulation under aseptic conditions. In some embodiments, provided methods further comprise diluting the first formulation within 1 day, 2 days, or 3 days prior to administering the second formulation.

In some embodiments, a first formulation of terevalefim comprises: about 10 mg/mL terevalefim;
about 400/(w/v) to about 60% (w/v) polyethylene glycol (e.g., polyethylene glycol 300);
about 5% (w/v) to about 15% (w/v) polysorbate (e.g., polysorbate 80); and
one or more aqueous components; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from aqueous buffer, phosphate buffered saline and normal saline; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from aqueous buffer, phosphate buffered saline and normal saline.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80); and
one or more aqueous components; and
   a second formulation of terevalefim comprises:
about 6 mg/mL terevalefim;
about 30/c (w/v) polyethylene glycol (e.g., polyethylene glycol 300);
about 6% (w/v) polysorbate (e.g., polysorbate 80); and
one or more aqueous components.

In some embodiments, a first formulation of terevalefim comprises:
about 10 mg/mL terevalefim;
about 50/c (w/v) polyethylene glycol (e.g., polyethylene glycol 300);
about 10% (w/v) polysorbate (e.g., polysorbate 80); and
one or more aqueous components selected from aqueous buffer, phosphate buffered saline and normal saline; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80); and
one or more aqueous components selected from aqueous buffer, phosphate buffered saline and normal saline.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80); and
one or more aqueous components selected from phosphate buffered saline and normal saline; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from aqueous buffer, phosphate buffered saline, and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from aqueous buffer, phosphate buffered saline, and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80);
one or more aqueous components; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80);
one or more aqueous components selected from aqueous buffer, phosphate buffered saline, and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from aqueous buffer, phosphate buffered saline, and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
compound 3 in an amount such that when a sample of the formulation is evaluated using HPLC (e.g., HPLC Method B or HPLC Method C of Example 7), the area percent of compound 3 is between about 0.05% and about 1.0%, or between about 0.05% and about 0.5%, or between about 0.05% and about 0.3%.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components; and
about 0.003 mg/mL to about 0.06 mg/mL, about 0.003 mg/mL to about 0.03 mg/mL, or about 0.003 mg/mL to about 0.018 mg/mL compound 3.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from aqueous buffer, phosphate buffered saline, and normal saline; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from aqueous buffer, phosphate buffered saline, and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, a first formulation of terevalefim comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 20% (w/v) to about 40% (w/v) polyethylene glycol (e.g., polyethylene glycol 300);
about 5% (w/v) to about 15% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, a first formulation of terevalefim comprises:
about 10 mg/mL terevalefim;
about 50% (w/v) polyethylene glycol (e.g., polyethylene glycol 300);
about 10% (w/v) polysorbate (e.g., polysorbate 80);
one or more aqueous components; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 300/a (w/v) polyethylene glycol (e.g., polyethylene glycol 300);
about 6% (w/v) polysorbate (e.g., polysorbate 80);
one or more aqueous components; and
about 0.003 mg/mL to about 0.06 mg/mL, about 0.003 mg/mL to about 0.03 mg/mL, or about 0.003 mg/mL to about 0.018 mg/mL compound 3.

In some embodiments, a first formulation of terevalefim comprises.
about 10 mg/mL terevalefim;
about 50% (w/v) polyethylene glycol (e.g., polyethylene glycol 300);
about 10% (w/v) polysorbate (e.g., polysorbate 80);
one or more aqueous components selected from aqueous buffer, phosphate buffered saline, and normal saline; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from aqueous buffer, phosphate buffered saline, and normal saline; and
about 0.003 mg/mL to about 0.06 mg/mL, about 0.003 mg/mL to about 0.03 mg/mL, or about 0.003 mg/mL to about 0.018 mg/mL compound 3.

In some embodiments, a first formulation of terevalefim comprises:
about 10 mg/mL terevalefim;
about 50% (w/v) polyethylene glycol (e.g., polyethylene glycol 300);
about 100 (w/v) polysorbate (e.g., polysorbate 80);
one or more aqueous components selected from phosphate buffered saline and normal saline; and
about 0.005 mg/mL to about 0.10 mg/mL, about 0.005 mg/mL to about 0.05 mg/mL, or about 0.005 mg/mL to about 0.03 mg/mL compound 3; and
   a second formulation of terevalefim comprises:
about 6 mg/mL terevalefim;
about 30% (w/v) polyethylene glycol (e.g., polyethylene glycol 300);
about 6% (w/v) polysorbate (e.g., polysorbate 80);
one or more aqueous components selected from phosphate buffered saline and normal saline; and
about 0.003 mg/mL to about 0.06 mg/mL, about 0.003 mg/mL to about 0.03 mg/mL, or about 0.003 mg/mL to about 0.018 mg/mL compound 3.

Many modifications and variations of the embodiments described herein may be made without departing from the scope, as is apparent to those skilled in the art. The specific embodiments described herein are offered by way of example only.

### EXAMPLES

### Example 1. Synthesis of Terevalefim

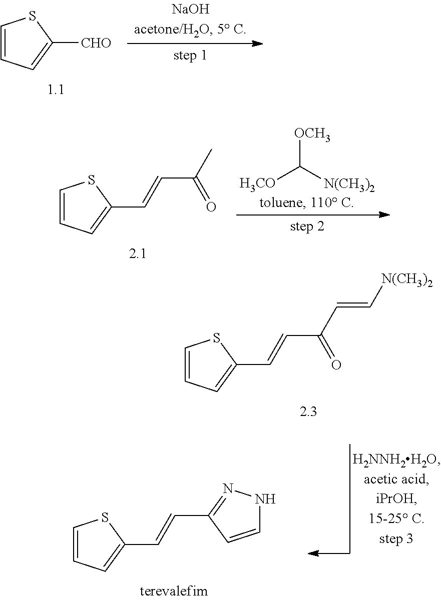

### Step 1. Synthesis of (E)-4-(thiophe-2-yl)but-3-en-2-one (2.1

To a solution of thiophene-2-carboxaldehyde (1.1, 40 kg) in 70 L of acetone was added 80 kg of 0.4 M aqueous NaOH slowly at 5° C. The solution was warmed to room temperature and stirred for 2-3 hours. Dichloromethane (approx. 4.5 volumes) was then added to the reaction mixture, the layers were allowed to separate, and the organic layer was removed. The aqueous layer was extracted with dichloromethane (approx. 0.7 volumes), the layers were allowed to separate, and the organic layer was combined with the first organic layer. Water (approx. 0.8 volumes) was added, the layers were allowed to separate, and the organic layer was combined with the first and second organic layers. The combined organic layers were then concentrated and diluted with toluene, which was then distilled with a Dean Stark apparatus to remove water. The mixture was then polish filtered and further concentrated to 3-4 volumes to provide a solution of (E)-4-(thiophen-2-yl)but-3-en-2-one (2.1) in toluene, which was carried forward into the next step without further purification.

### Step 2. Synthesis of (1E,4E)-(dimethylamino)-5-(thiophen-2-yl)penta-1,4-dien-3-one (2.3

To a solution of 2.1 in toluene from the previous step, N,N-dimethylformamide dimethylacetal (2.2a, 97 kg), was added and the reaction mixture heated at reflux for 36 hours. The reaction mixture was then concentrated to 2.5-3.5 volumes. The resulting slurry was cooled to room temperature and ethyl acetate was added. The resulting solids were filtered, washed with ethyl acetate, and dried under vacuum to afford (1E,4E)-1-(dimethylamino)-5-(thiophen-2-yl)penta-1,4-dien-3-one (2.3) in 55% yield from thiophene-2-carboxaldehyde (2.1).

In some instances, a slurry of seed material of 2.3 in ethyl acetate was added after concentration of the reaction mixture. Seed material of 2.3 can be prepared using the process described above.

### Step 3. Synthesis of Terevalefim

2.3 (40 kg) was dissolved in 130 kg of isopropyl alcohol under nitrogen, and the mixture was cooled to 10° C. Acetic acid (13 kg) was then added at 5-25° C. The solution was then cooled to 5-15° C., followed by slow addition of hydrazine hydrate (11.5 kg) while maintaining a temperature of 10-25° C. The reaction mixture was then stirred at 20° C. Once the reaction was judged to be complete (e.g., using HPLC or thin layer chromatography), water was added, and the resulting solids were filtered, washed with a mixture of isopropyl alcohol and water (1:4), and dried to afford crude terevalefim in 90% yield. Crude terevalefim obtained via this method was evaluated for compound 3 (280 nm: 0.28 area %; 220 nm: 0.12 area %) and Impurity A (220 nm: <0.05 area %) using HPLC Method B described in Example 7, among other things.

Crude terevalefim (26 kg) was dissolved in 42 kg acetonitrile at 65-75° C. and then slowly cooled to 15-25° C. to induce crystallization. The resulting solids were filtered and washed with a mixture of acetonitrile and water, and then dried under vacuum. The solids were then dissolved in ethyl acetate at 55-65° C. and polish filtered. n-Heptane (200 kg) was then added to the mixture at 50-60° C. The mixture was cooled to 15-25° C., the resulting solids were isolated by filtration, washed with n-heptane, and dried under vacuum to afford crystalline Form A terevalefim in 67% yield. Crystalline terevalefim obtained via this method was observed to contain between 3 and 25 ppm acetonitrile using Gas Chromatography Method A described in Example 7.

### Example 2. Alternative Synthesis of Terevalefim

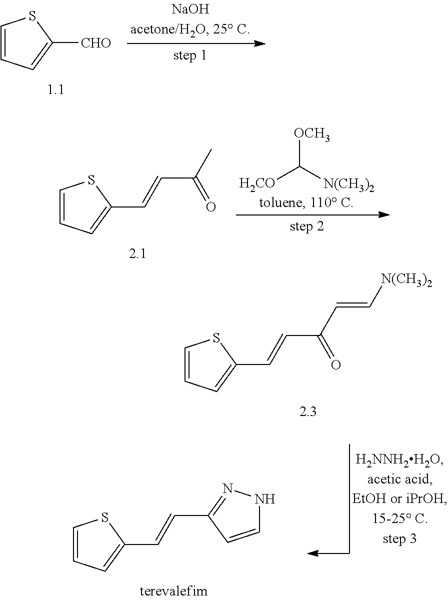

### Step 1. Synthesis of (E)-4-(thiophen-2-yl)but-3-en-2-one (2.1

To a solution of thiophene-2-carboxaldehyde (1.1, 23 kg) in 40 L of acetone was added 22 kg water and 0.75 kg of solid NaOH slowly at 25° C. The solution was stirred for 2-3 hours. The phases were allowed to separate. Dichloromethane (approx. 0.65 volumes) was then added to the aqueous phase, the layers were allowed to separate, and the organic layers were combined. The organic layers were then washed with aqueous brine (approx. 0.65 volumes). The organic layer was separated and dried with sodium sulfate (5 kg) and concentrated to a dry residue to give (E)-4-(thiophen-2-yl)but-3-en-2-one (2.1).

### Step 2. Synthesis of (1E,4E)-(dimethylamino)-S-(thiophen-2-yl)penta-1,4-dien-3-one (2.3

A solution of 2.1 (31 kg) in toluene (approx. 1.65 volumes) was combined with N,N-dimethylformamide dimethylacetal (2.2a, 52 kg), and the reaction mixture heated at reflux for 13 hours. The reaction mixture was then concentrated to near dryness under vacuum. The resulting oil was combined with 1:1 ethyl acetate:heptane and stirred. The resulting solids were filtered, washed with 1:1 ethyl acetate:heptane, and dried under vacuum to afford (1E,4E)-1-(dimethylamino)-5-(thiophen-2-yl)penta-1,4-dien-3-one (2.3) in 60% yield from thiophene-2-carboxaldehyde (1.1).

### Step 3. Synthesis of Terevalefim

2.3 (23 kg) was dissolved in 108 kg of ethyl alcohol under nitrogen, and hydrazine hydrate (7 kg) was added. Acetic acid (8.3 kg) was then added while maintaining a temperature of less than 30° C. The reaction mixture was then stirred at 30° C. Once the reaction was judged to be complete (e.g., using HPLC or thin layer chromatography), the reaction mixture was concentrated under vacuum, water was added, and the resulting solids were filtered, washed with a mixture of ethyl alcohol and water (1:4), and dried to afford crude terevalefim. Crude terevalefim (25.1 kg) was dissolved in 42 L acetonitrile at reflux and then slowly cooled to 0-10° C. to induce crystallization. The resulting solids were filtered and washed with cold acetonitrile, and then dried under vacuum to afford crystalline Form A terevalefim in 39/o yield.

An alternative method for the synthesis of terevalefim as described in Example 2 follows: 2.3 (35 kg) was dissolved in 300 kg of isopropyl alcohol under nitrogen, and hydrazine hydrate (20 kg) was added. Acetic acid (12 kg) was then added while maintaining a temperature of less than 30° C. The reaction mixture was then stirred at 30° C. Once the reaction was judged to be complete (e.g., using HPLC or thin layer chromatography), the reaction mixture was concentrated under vacuum, water was added, and the resulting solids were filtered, washed with a mixture of isopropyl alcohol and water, and dried to afford crude terevalefim. Crude terevalefim (25.1 kg) was dissolved in 370 L ethyl acetate, filtered over alumina, washed with ethyl acetate, and then concentrated under vacuum to remove about 500 L of solvent. The resulting mass was cooled and hexane (100 L) was added. The resulting solids were filtered and washed with cold hexane, and then dried under vacuum to afford crystalline Form A terevalefim in 59% yield.

### Example 3. Preparation and Characterization of Terevalefim Solid Forms

### General Methods:

### X-Ray Powder Diffraction (XRPD)

XRPD patterns of samples from scaled-up preparations were recorded at ambient temperature on a Bruker D8 Advance X-ray diffractometer (Karlsruhe, Germany) using Cu Kα radiation (λ=1.54 Å) at 40 kV, 40 mA passing through a Vario monochromator (Karlsruhe, Germany). The sample was loaded on a zero-background holder and gently pressed by a clean glass slide to ensure co-planarity of the powder surface with the surface of the holder. Data were collected in a continuous scan mode with a step size of 0.05° and dwell time of 1 s over an angular range of 3° to 40° 20. Obtained diffractograms were analyzed with DIFFRAC.EVA diffraction software (Bruker, Wisconsin, USA).

In some cases, the X-ray intensity data were measured on a Bruker D8 Eco diffractometer system equipped with a graphite monochromator and a Cu Kα Sealed tube (λ=1.54 Å). The sample was loaded in a polyimide capillary and collected data in transmission mode. Bruker's APEX3 software suite (Bruker, Wisconsin, USA) was used to collect and extract the intensity data. Obtained diffractograms were analyzed with TOPAS software (Bruker, Wisconsin, USA). Mercury 4.2.0 software (Build 257471, Cambridge Crystallographic Data Centre, UK) was used to calculate the XRPD patterns from single crystal data.

### Thermogravimetric Analysis (TGA)

TGA was performed using a Discovery TGA 5500 (TA® Instruments, New Castle, Delaware, USA) instrument operating with TRIOS software (Version 5.0). The sample was placed in an aluminum pan. The sample cell was purged with dry nitrogen at a flow rate of 15 mL/min. A heating rate of 10° C./min from 25° C. to desired temperature was used in all the experiments.

### Differential Scanning Calorimetry (DSC)

Conventional DSC experiments were performed using a Discovery DSC 250 (TA® Instruments, New Castle, Delaware, USA) instrument equipped with a refrigerated cooling system (RCS90) and operating with TRIOS software (Version 5.0). The sample cell was purged with dry nitrogen at a flow rate of 50 mL/min. Accurately weighed samples (2-5 mg) placed in TZero pans with a pin hole were scanned at a heating rate of 10° C./min over a temperature range of 25° C. to desired temperature was used in all the experiments

### Terevalefim Lot I

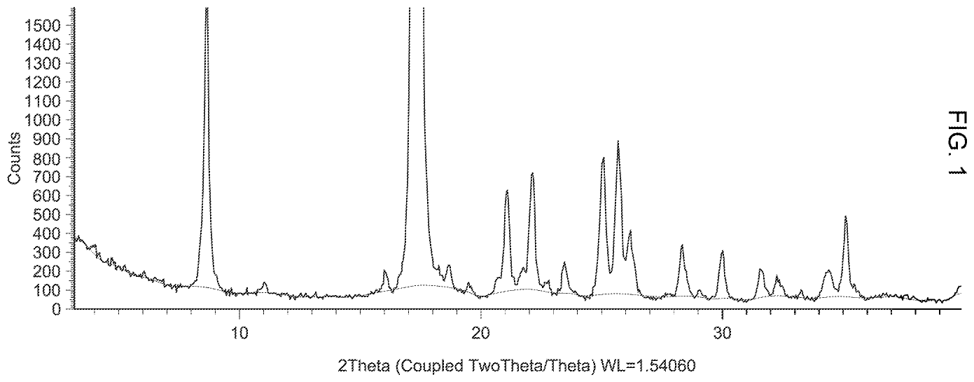

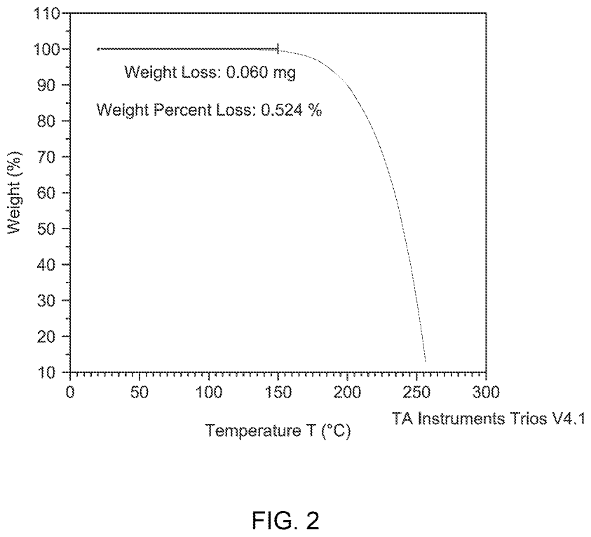

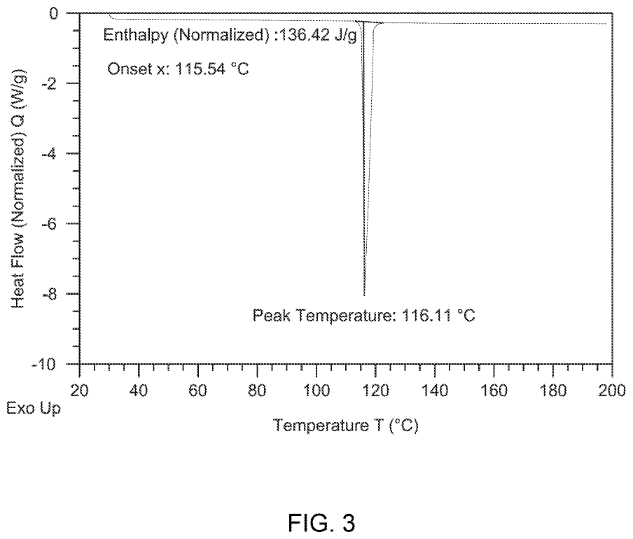

Terevalefim was provided (e.g., via the method of Example 2) in a form with an XRPD as shown in FIG. 1, a TGA as shown in FIG. 2, and a DSC as shown in FIG. 3. Herein, this material is referred to as “Terevalefim Lot I.”

### Form A

Terevalefim Form A was synthesized by recrystallizing Terevalefim Lot I from methanol. In a typical reaction, ˜450 mg of Terevalefim Lot I was dissolved in 2 mL of methanol while heating at 50° C. Resultant solution was kept at room temperature and allowed for slow evaporation of the solvent. Crystals suitable for single crystal X-ray diffraction were obtained within one day.

Terevalefim Form A bulk powder was prepared as follows: ˜ 5 g of Terevalefim Lot I was suspended in 5 mL of methanol and slurried at room temperature for two days. The resulting solid was filtered using 0.45 μm PTFE syringe filter.

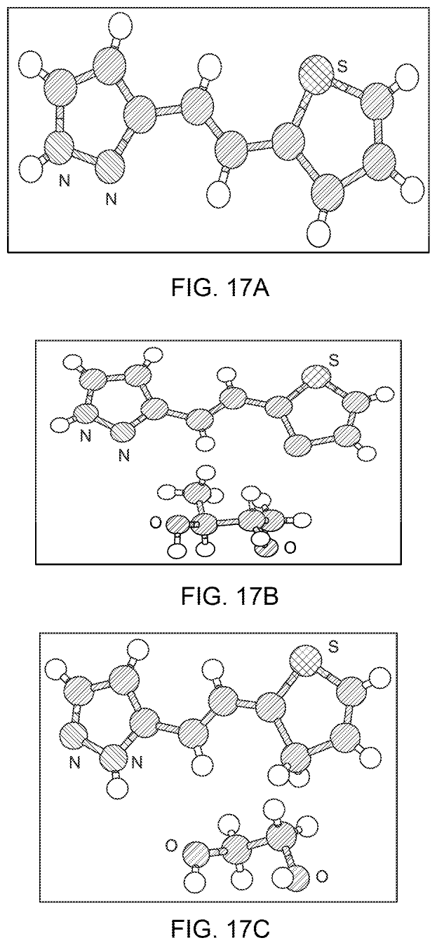

Single crystal X-ray diffraction of terevalefim Form A was obtained (FIG. 17A). Crystal data and structure refinement parameters are summarized below:

| | |
|---|---|
| Temperature (K) | 278.15 |
| Crystal system | Monoclinic |
| Space group | C2/c |
| a (Å) | 21.5572(10) |
| b (Å) | 4.9949(2) |
| c (Å) | 16.8890(8) |
| α (°) | 90 |
| β (°) | 108.445(2) |
| γ (°) | 90 |
| Volume (Å³) | 1725.12(14) |

The XRPD pattern of terevalefim Form A calculated from single crystal X-ray diffraction data is shown in FIG. 4 and is summarized below:

| 2θ (°) | d-spacing (Å) | Intensity Counts (I) |
|---|---|---|
| 8.64 | 10.22 | 1690 |
| 11.04 | 8.01 | 422.5 |
| 11.67 | 7.57 | 88.3 |
| 16.06 | 5.51 | 380.0 |
| 17.34 | 5.11 | 9972 |
| 18.27 | 4.85 | 464.7 |
| 18.69 | 4.74 | 1532 |
| 19.49 | 4.55 | 387.5 |
| 20.66 | 4.30 | 587.9 |
| 21.09 | 4.21 | 3023 |
| 21.70 | 4.09 | 1257 |
| 22.10 | 4.02 | 5053 |
| 22.76 | 3.90 | 636.7 |
| 23.46 | 3.79 | 723.6 |
| 23.74 | 3.74 | 238.5 |
| 25.06 | 3.55 | 6064 |
| 25.70 | 3.46 | 7656 |
| 26.12 | 3.41 | 301.6 |
| 26.32 | 3.38 | 352.7 |
| 27.64 | 3.23 | 147.8 |
| 27.78 | 3.21 | 140.2 |
| 28.31 | 3.15 | 2450 |
| 28.49 | 3.13 | 300.2 |
| 29.04 | 3.07 | 293.7 |
| 29.95 | 2.98 | 1525 |
| 31.59 | 2.83 | 197.4 |
| 31.82 | 2.81 | 125.7 |
| 32.25 | 2.77 | 707.3 |
| 33.22 | 2.69 | 71.4 |
| 34.21 | 2.62 | 391.5 |
| 34.42 | 2.60 | 415.4 |
| 35.08 | 2.56 | 291.9 |
| 35.53 | 2.52 | 173.3 |
| 36.33 | 2.47 | 100.8 |
| 36.70 | 2.45 | 316.9 |
| 37.16 | 2.42 | 309.8 |
| 37.65 | 2.39 | 53.2 |
| 39.02 | 2.31 | 22.1 |
| 39.60 | 2.27 | 156.6 |
| 39.81 | 2.26 | 392.5 |

The XRPD pattern of terevalefim Form A bulk powder is shown in FIG. 5. Comparison of observed XRPD pattern of terevalefim Form A bulk powder corresponds well with the calculated XRPD pattern (FIG. 4).

TGA of terevalefim Form A is shown in FIG. 6. A weight loss of 0.6% was observed up to 150° C.

DSC of terevalefim Form A is shown in FIG. 7. One endotherm was observed at 116.42° C.

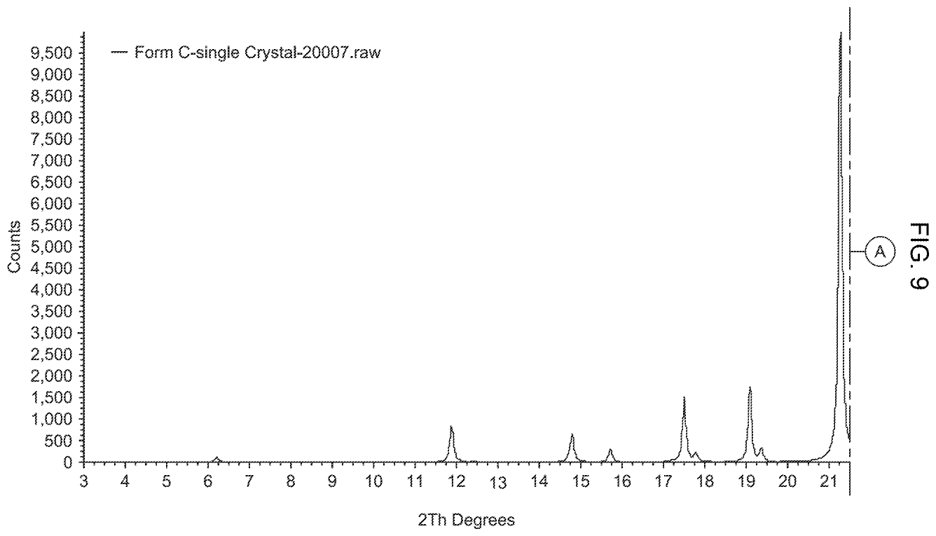

A comparison of Terevalefim Lot I is shown in FIG. 8 and verifies that Terevalefim Lot I matches Form A.

### Form C

Terevalefim Form C was prepared as follows: ˜500 mg of Terevalefim Lot I was dissolved in a solvent mixture (250 μL of propylene glycol and 1500 μL of methyl isobutyl ketone) while heating at 50° C. The resultant solution was kept at −27° C. for 48 hours to yield terevalefim Form C. Solids were collected by filtration using 0.45 μm PTFE syringe filter.

Single crystal X-ray diffraction of terevalefim Form C was obtained and showed that terevalefim Form C is a propylene glycol solvate (2:1 terevalefim:propylene glycol) (FIG. 17B). Crystal data and structure refinement parameters are summarized below:

| | |
|---|---|
| Temperature (K) | 278.15 |
| Crystal system | Orthorhombic |
| Space group | Pbcn |
| a (Å) | 28.481(4) |
| b (Å) | 7.7056(9) |
| c (Å) | 10.1243(13) |
| α (°) | 90 |
| β (°) | 90 |
| γ (°) | 90 |
| Volume (Å³) | 2221.9(5) |

The XRPD pattern of terevalefim Form C calculated from single crystal X-ray diffraction data is shown in FIG. 9 and is summarized below:

| 2θ (°) | d-spacing (Å) | Intensity Counts (I) |
|---|---|---|
| 6.20 | 14.24 | 115.6 |
| 11.88 | 7.44 | 816.6 |
| 12.41 | 7.13 | 31.7 |
| 14.79 | 5.99 | 645.4 |
| 15.72 | 5.63 | 294.4 |
| 17.50 | 5.06 | 1490 |
| 17.78 | 4.98 | 226.8 |
| 19.08 | 4.65 | 1725 |
| 19.36 | 4.58 | 327.8 |
| 21.28 | 4.17 | 10000 |
| 21.89 | 4.06 | 178.0 |
| 23.07 | 3.85 | 2644 |
| 23.69 | 3.75 | 338.2 |
| 23.92 | 3.72 | 68.5 |
| 24.70 | 3.60 | 418.4 |
| 24.90 | 3.57 | 191.4 |
| 25.50 | 3.49 | 96.2 |
| 25.71 | 3.46 | 154.9 |
| 26.28 | 3.39 | 646.2 |
| 27.74 | 3.21 | 464.2 |
| 28.13 | 3.17 | 81.9 |
| 29.10 | 3.07 | 81.6 |
| 29.31 | 3.04 | 132.3 |
| 29.55 | 3.02 | 209.9 |
| 30.51 | 2.93 | 215.7 |
| 31.15 | 2.87 | 1058 |
| 31.76 | 2.82 | 67.2 |
| 32.93 | 2.72 | 93.6 |
| 33.19 | 2.70 | 163.7 |
| 33.36 | 2.68 | 70.1 |
| 34.63 | 2.59 | 12.8 |
| 35.05 | 2.56 | 93.6 |
| 35.40 | 2.53 | 274.8 |
| 35.90 | 2.50 | 112.6 |
| 36.20 | 2.48 | 77.1 |
| 36.60 | 2.45 | 78.3 |
| 37.57 | 2.39 | 105.6 |
| 37.69 | 2.38 | 214.3 |
| 38.05 | 2.36 | 105.1 |
| 38.54 | 2.33 | 127.0 |
| 38.80 | 2.32 | 124.8 |
| 39.05 | 2.30 | 155.2 |
| 39.46 | 2.28 | 50.0 |
| 39.82 | 2.26 | 34.2 |

The XRPD pattern of terevalefim Form C collected from the scaled-up preparation is shown in FIG. 10. Comparison of observed XRPD pattern of terevalefim Form C corresponds well with the calculated XRPD pattern (FIG. 9).

TGA of terevalefim Form C is shown in FIG. 11. A weight loss of 20.27% was observed up to 150° C. Observed weight loss correlated well with calculated percentage of propylene glycol in crystal lattice based on single crystal X-ray diffraction (17.76%).

DSC of terevalefim Form C is shown in FIG. 12. One endotherm was observed at 75.22° C.

### Form D

Terevalefim Form D was prepared as follows: ˜500 mg of Terevalefim Lot I was dissolved in a solvent mixture (250 μL of ethylene glycol and 1500 μL of methyl isobutyl ketone) while heating at 50° C. The resultant solution was kept at −27° C. for 48 hours to yield terevalefim Form D. Solid was collected by filtration using 0.45 μm PTFE syringe filter.

Single crystal X-ray diffraction of terevalefim Form D was obtained and showed that terevalefim Form D is an ethylene glycol solvate (2:1 terevalefim:ethylene glycol) (FIG. 17C). Crystal data and structure refinement parameters are summarized below:

| | |
|---|---|
| Temperature (K) | 278.15 |
| Crystal system | Orthorhombic |
| Space group | Pbcn |
| a (Å) | 28.544(3) |
| b (Å) | 7.4452(8) |
| c (Å) | 9.8184(10) |
| α (°) | 90 |
| β (°) | 90 |
| γ (°) | 90 |
| Volume (Å³) | 2086.6(4) |

The XRPD pattern of terevalefim Form D calculated from single crystal X-ray diffraction data is shown in FIG. 13 and is summarized below:

| 2θ (°) | d-spacing (Å) | Intensity Counts (I) |
|---|---|---|
| 6.19 | 14.28 | 6185 |
| 12.28 | 7.20 | 1019 |
| 12.38 | 7.14 | 769.5 |
| 15.10 | 5.86 | 584.0 |
| 15.24 | 5.81 | 638.3 |
| 16.17 | 5.48 | 206.6 |
| 17.60 | 5.03 | 118.4 |
| 18.06 | 4.91 | 2066 |
| 18.63 | 4.76 | 175.5 |
| 19.11 | 4.64 | 56.9 |
| 19.44 | 4.56 | 774.4 |
| 20.35 | 4.36 | 51.4 |
| 21.58 | 4.11 | 10000 |
| 21.95 | 4.05 | 334.6 |
| 22.55 | 3.94 | 349.9 |
| 23.88 | 3.72 | 3614 |
| 24.70 | 3.60 | 97.3 |
| 24.88 | 3.58 | 260.0 |
| 25.57 | 3.48 | 60.9 |
| 25.76 | 3.46 | 70.1 |
| 26.07 | 3.42 | 88.3 |
| 26.33 | 3.38 | 141.1 |
| 26.50 | 3.36 | 646.6 |
| 26.75 | 3.33 | 100.4 |
| 26.99 | 3.30 | 52.9 |
| 27.25 | 3.27 | 148.4 |
| 28.51 | 3.13 | 925.8 |
| 28.72 | 3.11 | 94.2 |
| 29.96 | 2.98 | 44.2 |
| 30.10 | 2.97 | 72.2 |
| 30.26 | 2.95 | 54.3 |
| 30.47 | 2.93 | 305.5 |
| 30.62 | 2.92 | 204.7 |
| 30.75 | 2.90 | 119.6 |
| 31.27 | 2.86 | 69.2 |
| 31.57 | 2.83 | 62.0 |
| 31.83 | 2.81 | 617.8 |
| 31.97 | 2.80 | 259.2 |
| 32.38 | 2.76 | 59.6 |
| 32.67 | 2.74 | 83.2 |
| 33.61 | 2.66 | 112.6 |
| 33.82 | 2.65 | 233.2 |
| 34.84 | 2.57 | 17.0 |
| 35.78 | 2.51 | 344.8 |
| 36.02 | 2.49 | 92.8 |
| 36.36 | 2.47 | 95.0 |
| 36.53 | 2.46 | 110.3 |
| 36.66 | 2.45 | 98.4 |
| 37.10 | 2.42 | 203.5 |
| 37.44 | 2.40 | 74.6 |
| 37.81 | 2.38 | 129.3 |
| 38.38 | 2.34 | 47.4 |
| 38.72 | 2.32 | 499.3 |
| 39.24 | 2.29 | 300.7 |
| 39.78 | 2.26 | 61.9 |

The XRPD pattern of terevalefim Form D collected from the scaled-up preparation is shown in FIG. 14. Comparison of observed XRPD pattern of terevalefim Form D corresponds well with the calculated XRPD pattern (FIG. 13).

TGA of terevalefim Form D is shown in FIG. 15. A weight loss of 16.46% was observed up to 150° C. Observed weight loss correlated well with calculated percentage of ethylene glycol in crystal lattice based on single crystal X-ray diffraction (14.97%).

DSC of terevalefim Form D is shown in FIG. 16. One endotherm was observed at 76.95° C.

### Example 4. Preparation of a Terevalefim Formulation

Polyethylene glycol 300 (175 kg) and polysorbate 80 (35 kg) were combined in a 440 L vessel. Terevalefim (3.5 kg, adjusted for purity, water content, and residual solvent) was added and stirred for 60 minutes. Phosphate buffered saline was filtered and added to the solution until the total mass of the compounding mixture was 381.5 kg. 1.0 N HCl and/or 1.0 N NaOH was added to bring pH to 7.5-7.9. The resulting solution was filtered through 0.45 μm and 0.22 μm polish filters in succession, then filled into 20 mL Type I glass vials with a target fill weight of 25.12 g per vial and nitrogen over-fill. Vials were capped with B2-40 West stoppers and sealed. This procedure provided a 10 mg/mL terevalefim formulation.

Phosphate buffered saline used in this Example was prepared as follows: 0.2 mg/mL potassium chloride, 0.2 mg/mL monobasic potassium phosphate, 8.0 mg/mL sodium chloride, and 2.16 mg/mL sodium phosphate dibasic heptahydrate were dissolved in water (e.g., water for injection). The pH was then adjusted as needed using 1.0 N HCl (aq) and 1.0 N NaOH (aq).

A 6 mg/mL terevalefim formulation was prepared by diluting the 10 mg/mL formulation described above with normal saline.

### Example 5. Stability of Provided Formulations

Two formulations of terevalefim were evaluated for stability at various conditions. Formulation A and Formulation B were prepared as described in Table 1.

TABLE 1

| Component | Formulation A | Formulation B | |
|---|---|---|---|
| Terevalefim | 10 mg/mL | 6 mg/mL | |
| Polyethylene | 50% (w/v) | 30% | (w/v) |
| glycol 300 | | | |
| Polysorbate 80 | 10% (w/v) | 6% | (w/v) |

| | | |
|---|---|---|
| Aqueous Components | 40% (w/v) phosphate | 24% (w/v) phosphate |
| | buffered saline | buffered saline and |
| | | 40% (w/v) normal saline |

Stability results for Formulation A stored upright at −20° C. in a USP Type I glass vial are summarized in Table 2.

TABLE 2

| Test | Initial | 3 Mo. | 6 Mo. | 9 Mo. | 12 Mo. | 18 Mo. | 24 Mo. | 36 Mo. | 48 Mo. | 60 Mo. |
|---|---|---|---|---|---|---|---|---|---|---|
| Color | SY^{a} | SY^{a} | SY^{a} | SY^{a} | SY^{a} | SY^{a} | SY^{a} | SY^{a} | SY^{a} | SY^{a} |
| pH | 8 | 8 | 8 | 8 | 8 | 8 | 8 | 8 | 8 | 9 |
| Assay^{b} (%) | 101.4 | 105.9 | 104.7 | 103.6 | 103.2 | 103.6 | 103.5 | 104.3 | 103.2 | 104.2 |
| HPLC^{c} | 0.26 | 0.30 | 0.27 | 0.27 | 0.27 | 0.25 | 0.31 | 0.26 | 0.25 | 0.26 |
| (Cmpd 3, | | | | | | | | | | |
| area %) | | | | | | | | | | |
| HPLC^{c} | — | — | — | — | — | — | — | — | 0.12 | — |
| (Cmpd 1.1, | | | | | | | | | | |
| area %) | | | | | | | | | | |
| HPLC^{c} | 0.26 | 0.30 | 0.27 | 0.27 | 0.27 | 0.32 | 0.35 | 0.34 | 0.37 | 0.34 |
| (total | | | | | | | | | | |
| impurities, | | | | | | | | | | |
| area %) | | | | | | | | | | |
| Particulate | 1 | — | — | — | 0 | — | 0 | 140 | 87 | 84 |
| Matter | | | | | | | | | | |
| (≥10 μm) | | | | | | | | | | |
| Particulate | 0 | — | — | — | 0 | — | 0 | 40 | 8 | 16 |
| Matter | | | | | | | | | | |
| (≥25 μm) | | | | | | | | | | |
| Sterility | Pass | — | — | — | Pass | — | Pass | Pass | Pass | Pass |

^{a}SY = slightly yellow;
^{b}Amount terevalefim as percentage of initial amount added;
^{c}Measured using HPLC Method C of Example 7.

Stability results of Formulation A stored upright at 5° C. in a USP Type I glass vial are summarized in Table 3.

TABLE 3

| Test | Initial | 2 Mo. | 3 Mo. | 6 Mo. | 60 Mo. |
|---|---|---|---|---|---|
| Color | SY^{a} | SY^{a} | SY^{a} | SY^{a} | SY^{a} |
| pH | 8 | 8 | | 8 | 8 |
| Assay^{b} (%) | 101.4 | 103.4 | 102.4 | 102.6 | 100.4 |
| HPLC^{c} (Cmpd 3, area %) | 0.26 | 0.25 | 0.24 | 0.26 | 0.19 |
| HPLC^{c} (Cmpd 1.1, | — | — | — | 0.10 | 0.38 |
| area %) | | | | | |
| HPLC^{c} (total impurities, | 0.26 | 0.25 | 0.33 | 0.36 | 0.56 |
| area %) | | | | | |
| Particulate Matter | 1 | — | — | 1 | — |
| (≥10 μm) | | | | | |
| Particulate Matter | 0 | — | — | 0 | — |
| (≥25 μm) | | | | | |
| Sterility | Pass | — | — | — | Pass |

^{a}SY = slightly yellow;
^{b}Amount terevalefim as percentage of initial amount added;
^{c}Measured using HPLC Method C of Example 7.

Stability results of Formulation A stored upright at 25° C. in a USP Type I glass vial are summarized in Table 4.

TABLE 4

| Test | Initial | 1 Mo. | 3 Mo. | 6 Mo. |
|---|---|---|---|---|
| Color | VLY^{a} | SY^{b} | SY^{b} | SY^{b} |
| pH | 8.1 | 7.9 | 7.8 | 7.3 |
| Assay^{c} (%) | 103.4 | 103.4 | 102.4 | 103.5 |
| HPLC^{d} (Cmpd 3, area %) | 0.25 | 0.30 | 0.30 | 0.25 |
| HPLC^{d} (Cmpd 1.1, area %) | — | — | — | 0.29 |
| HPLC^{d} (total impurities, | 0.25 | 0.35 | 0.50 | 0.62 |
| area %) | | | | |
| Particulate Matter | 1584 | 37 | 107 | 12 |
| (≥10 μm) | | | | |
| Particulate Matter | 139 | 26 | 87 | 8 |
| (≥25 μm) | | | | |

^{a}VLY = very light yellow;
^{b}SY = slightly yellow;
^{c}Amount terevalefim as percentage of initial amount added;
^{d}Measured using HPLC Method C of Example 7.

Stability results of Formulation A stored upright at −70° C. in a USP Type I glass vial are summarized in Table 5.

TABLE 5

| Test | Initial | 1 Wk. | 1 Mo. | 3 Mo. |
|---|---|---|---|---|
| Color | VLY^{a} | SY^{b} | SY^{b} | SY^{b} |
| pH | 7.7 | 8.1 | 8.1 | 8.1 |
| Assay^{c} (%) | 104.7 | 103.6 | 102.8 | 105.8 |
| HPLC^{d} (total impurities, | 0.36 | 0.36 | 0.36 | 0.36 |
| area %) | | | | |
| Particulate Matter | 1131 | 1 | 894 | 25 |
| (≥10 μm) | | | | |
| Particulate Matter | 94 | <1 | 143 | 8 |
| (≥25 μm) | | | | |

^{a}VLY = very light yellow;
^{b}SY = slightly yellow;
^{c}Amount terevalefim as percentage of initial amount added;
^{d}Measured using HPLC Method C of Example 7.

Stability results of Formulation B stored upright at 5° C. in a USP Type I glass vial are summarized in Table 6.

TABLE 6

| Test | Initial | 1 Mo. | 2 Mo. | 3 Mo. | 6 Mo. |
|---|---|---|---|---|---|
| Color | CC^{a} | LY^{a} | LY^{a} | LT^{a} | LA^{a} |
| pH | 6.7 | 6.5 | 6.4 | 6.3 | 6.1 |
| Assay^{b} (%) | 99.1 | 100.5 | 99.2 | 98.4 | 98.8 |
| HPLC^{c} (Cmpd 3, area %) | 0.25 | 0.25 | 0.27 | 0.26 | 0.28 |
| HPLC^{c} (Cmpd 1.1, | 0.09 | 0.22 | — | 0.39 | 0.58 |
| area %) | | | | | |
| HPLC^{c} (total impurities, | 0.34 | 0.47 | 0.69 | 0.65 | 1.05 |
| area %) | | | | | |
| Particulate Matter | 378 | 662 | — | — | 31 |
| (≥10 μm) | | | | | |
| Particulate Matter | 30 | 8 | — | — | 0 |
| (≥25 μm) | | | | | |

^{a}CC = clear, colorless; LY = light yellow; LT = light tan; LA = light amber;
^{b}Amount terevalefim as percentage of initial amount added;
^{c}Measured using HPLC Method B of Example 7.

Stability results of Formulation B stored upright at 25° C./60% RH in a USP Type glass vial are summarized in Table 7.

TABLE 7

| Test | 1 Mo. | 2 Mo. | 3 Mo. | 6 Mo. |
|---|---|---|---|---|
| Color | A^{a} | LT^{a} | LA^{a} | A^{a} |
| pH | 5.8 | 5.6 | 5.4 | 5.1 |
| Assay^{b} (%) | 97.2 | 95.7 | 93.6 | 91.9 |
| HPLC^{c} (Cmpd 3, area %) | 0.25 | 0.27 | 0.25 | 0.27 |
| HPLC^{c} (Cmpd 1.1, area %) | 0.55 | — | 1.11 | 1.33 |
| HPLC^{c} (total impurities, | 0.86 | 1.33 | 1.41 | 1.91 |
| area %) | | | | |
| Particulate Matter | 1135 | — | — | 73 |
| (≥10 μm) | | | | |
| Particulate Matter | 2 | — | — | 0 |
| (≥25 μm) | | | | |

^{a}A = amber; LT = light tan; LA = light amber;
^{b}Amount terevalefim as percentage of initial amount added;
^{c}Measured using HPLC Method B of Example 7.

Stability results of Formulation B stored upright at 40° C./75% RH in a USP Type I glass vial are summarized in Table 8.

TABLE 8

| Test | 1 Mo. | 2 Mo. | 3 Mo. | 6 Mo. |
|---|---|---|---|---|
| Color | A^{a} | LT^{a} | LA^{a} | A^{a} |
| pH | 5.1 | 5.0 | 4.9 | 4.8 |
| Assay^{b} (%) | 94.0 | 91.7 | 90.5 | 91.0 |
| HPLC^{c} (Cmpd 3, area %) | 0.26 | 0.27 | 0.25 | 0.28 |
| HPLC^{c} (Cmpd 1.1, area %) | 0.99 | — | 1.37 | 1.52 |
| HPLC^{c} (total impurities, | 1.38 | 1.76 | 1.74 | 2.10 |
| area %) | | | | |
| Particulate Matter | 8809 | — | — | 96 |
| (≥10 μm) | | | | |
| Particulate Matter | 818 | — | — | 0 |
| (≥25 μm) | | | | |

^{a}A = amber; LT = light tan; LA = light amber;
^{b}Amount terevalefim as percentage of initial amount added;
^{c}Measured using HPLC Method B of Example 7.

### Example 6. Evaluation of Hydrazine Content in Preparation of Terevalefim

The following experiment demonstrates effectiveness of certain synthetic steps in reducing the amount of hydrazine in compositions of terevalefim, e.g., as produced by Example 1. Hydrazine content from each sample described below was analyzed using HPLC according to the following HPLC Method A of Example 7.

Sample 1: Prepared as described in Example 1, Step 3, except that once the reaction was judged to be complete (e.g., using HPLC or thin layer chromatography), the reaction mixture was concentrated to dryness to give a residue.

Sample 2: Prepared as described for Sample 1 with additional steps as follows: After reaction was judged to be complete, water was added, and the resulting solids were filtered and washed with a mixture of isopropyl alcohol and water (1:5) (3 volumes).

Sample 3: Prepared according to Example 1, Step 3 through acetonitrile recrystallization, except acetonitrile spiked with 1.6 wt % hydrazine hydrate was used for recrystallization.

Sample 4: Prepared according to Example 1, Step 3 through acetonitrile recrystallization, except acetonitrile spiked with 0.16 wt % hydrazine hydrate was used for recrystallization.

Sample 5: Prepared according to Example 1, Step 3, except ethyl acetate spiked with 1.6 wt % hydrazine hydrate was used in ethyl acetate recrystallization step.

Sample 6: Prepared according to Example 1, Step 3, except ethyl acetate spiked with 0.16 wt % hydrazine hydrate was used in ethyl acetate recrystallization step.

Sample 7: Prepared as described for Sample 3, and solids carried through remaining steps of Example 1, Step 3.

Sample 8: Prepared as described for Sample 4, and solids carried through remaining steps of Example 1, Step 3.

Results from hydrazine analysis of Samples 1-8 are summarized in Table 9 and demonstrate that provided methods, such as those described in Example 1, are effective at reducing hydrazine content below acceptable limits.

TABLE 9

| Sample | Hydrazine Content (ppm) |
|---|---|
| 1 | 20 |
| 2 | 1 |
| 3 | 4 |
| 4 | 4 |
| 5 | <3 |
| 6 | <3 |
| 7 | <3 |
| 8 | <3 |

### Example 7. Analytical Methods

Gas Chromatography Method A

Gas chromatography performed according to the following parameters was used to determine residual solvent (e.g., acetonitrile) concentrations in samples obtained from, e.g., the synthesis of Example 1.

| Column | DB-624, 60 m × 0.32 mm × 1.8 μm film | | |
|---|---|---|---|
| Injection Liner | 4 mm straight splitless liner | | |
| Inlet Temperature | 250° C. | | |
| Carrier Gas Flow Rate | 1.5 mL/min | | |
| Detector | FID | | |
| Split Ratio | 5:1 | | |
| Detector Temperature | 300° C. | | |
| Hydrogen Flow | 45 mL/min | | |
| Air Flow | 450 mL/min | | |
| Make Up Gas (N₂ or He) | 25 mL/min | | |
| Aux Pressure | 15 psi | | |
| Run Time | 33 min | | |
| Over Temperature | Rate | Temp. | Hold time |
| Program | (° C./min) | (° C.) | (min) |
| | Initial | 40 | 10 |
| | 20 | 80 | 2 |
| | 10 | 220 | 5 |

### HPLC Method A

High performance liquid chromatography performed according to the following parameters was used to determine hydrazine content in samples obtained from, e.g., the synthesis of Example 1.

| Column | Phenomenex Luna C18, 250 mm × 4.6 | | |
|---|---|---|---|
| | mm, 5 μm with MacMod column saver pre- | | |
| | filter, 0.5 μm, PN: MMCS210/HI686 | | |
| Detection Type | UV | | |
| Detection Wavelength | 300 nm | | |
| Column Temperature | Ambient | | |
| Autosampler Temperature | Ambient | | |
| Flow Rate | 1.00 mL/min | | |
| Injection Volume | 10.0 μL | | |
| Approximate Run Time | 40 min | | |
| Mobile Phase A | 0.1% trifluoroacetic acid in water | | |
| Mobile Phase B | 0.1% trifluoroacetic acid in acetonitrile | | |
| Gradient | Time (min) | % A | % B |
| | 0.0 | 80 | 20 |
| | 20.0 | 20 | 80 |
| | 30.0 | 20 | 80 |
| | 31.0 | 80 | 20 |
| | 40.0 | 80 | 20 |

### HPLC Method B

High performance liquid chromatography performed according to the following parameters was used to analyze samples of terevalefim, e.g., samples obtained from the synthesis of Example 1.

| Column | Phenomenex Luna C18, 250 mm × 4.6 | | |
|---|---|---|---|
| | mm, 5 μm | | |
| Detection Type | UV | | |
| Detection Wavelength | 280 nm | | |
| | 220 nm | | |
| Column Temperature | 25° C. | | |
| Autosampler Temperature | Ambient | | |
| Flow Rate | 1.0 mL/min | | |
| Injection Volume | 10 μL | | |
| Approximate Run Time | 40 min | | |
| Mobile Phase A | 0.1% trifluoroacetic acid in water | | |
| Mobile Phase B | 0.1% trifluoroacetic acid in acetonitrile | | |
| Gradient | Time (min) | % A | % B |
| | 0.0 | 80 | 20 |
| | 20.0 | 20 | 80 |
| | 30.0 | 20 | 80 |
| | 31.0 | 80 | 20 |
| | 40.0 | 80 | 20 |

### HPLC Method C

High performance liquid chromatography performed according to the following parameters was used to analyze samples of terevalefim, e.g., samples obtained from the synthesis

| Column | C18, 150 mm × 4.6 mm, 5 μm | |
|---|---|---|
| Detection Type | UV | |
| Detection Wavelength | 280 nm | |
| Column Temperature | 25° C. | |
| Sample Temperature | 5° C. | |
| Flow Rate | 0.1 mL/min | |
| Injection Volume | 10 μL | |
| Approximate Run Time | 10 min | |
| Mobile Phase A | 0.1% trifluoroacetic acid in water | |
| Mobile Phase B | 0.1% trifluoroacetic acid in acetonitrile | |
| Mobile Phase Ratio | % A | % B |
| | 65 | 35 |

### Example 8. Alternative Preparation of a Terevalefim Formulation

Polyethylene glycol 300 (0.6 kg) and polysorbate 80 (0.12 kg) were combined in a vessel. Terevalefim (0.012 kg, adjusted for purity, water content, and residual solvent) was added and stirred until dissolved. A portion of water (e.g., water for injection) was added to the mixture, equal to approximately half the expected quantity. Then, monobasic potassium phosphate (0.00176 kg) was added, followed by dibasic sodium phosphate (0.00726 kg). The pH was then adjusted as needed using 1.0 N HCl (aq) and 1.0 N NaOH (aq) to achieve a pH of 7.7 f 0.2. Water (e.g., water for injection) was added to bring the terevalefim concentration to 6 mg/mL. This Example provides a formulation comprising: about 6 mg/mL terevalefim, about 30% (w/v) polyethylene glycol 300, about 6% (w/v) polysorbate 80, about 0.07% (w/v) monobasic potassium phosphate, about 0.4% (w/v) dibasic sodium phosphate, and about 63% (w/v) water.

### Example 9. Stability of Provided Formulations

A formulation of terevalefim was evaluated for stability at various conditions. Formulation C was prepared as described in Table 10.

TABLE 10

| Component | Formulation C | |
|---|---|---|
| Terevalefim | 6 mg/mL | |
| Polyethylene glycol 300 | 30% | (w/v) |
| Polysorbate 80 | 6% | (w/v) |

| | |
|---|---|
| Aqueous Components | 64% (w/v) aqueous |
| | phosphate buffer |

Stability results for Formulation C stored upright at 5° C. in a 20 mL glass vial are summarized in Table 11.

TABLE 11

| Test | 0 Mo. | 1 Mo. |
|---|---|---|
| Color | SY^{a} | SY^{a} |
| pH | 7.8 | 7.8 |
| Assay^{b} (%) | 101.2 | 99.7 |
| HPLC^{c} (Cmpd 3, area %) | 0.31 | 0.30 |
| HPLC^{c} (Cmpd 1.1, area %) | <0.05 | 0.06 |
| HPLC^{c} (total impurities, area %) | 0.31 | 0.36 |
| Particulate Matter (≥10 μm) | 21 | 56 |
| Particulate Matter (≥25 μm) | 0 | 1 |

^{a}SY = slightly yellow;
^{b}Amount terevalefim as percentage of initial amount added;
^{c}Measured using HPLC Method B of Example 7.

Stability results for Formulation C stored upright at 25° C./60/o RH in a 20 mL glass vial are summarized in Table 12.

TABLE 12

| Test | 1 Mo. |
|---|---|
| Color | SY^{a} |
| pH | 7.8 |
| Assay^{b} (%) | 98.9 |
| HPL^{c} (Cmpd 3, area %) | 0.30 |
| HPLC^{c} (Cmpd 1.1, area %) | 0.10 |
| HPLC^{c} (total impurities, area %) | 0.40 |
| Particulate Matter (≥10 μm) | 63 |
| Particulate Matter (≥25 μm) | 0 |

^{a}SY = slightly yellow;
^{b}Amount terevalefim as percentage of initial amount added;
^{c}Measured using HPLC Method B of Example 7.

Stability results for Formulation C stored upright at 40° C./75% RH in a 20 mL glass vial are summarized in Table 13.

TABLE 13

| Test | 1 Mo. |
|---|---|
| Color | SY^{a} |
| pH | 7.7 |
| Assay^{b} (%) | 98.6 |
| HPLC^{c} (Cmpd 3, area %) | 0.30 |
| HPL^{c} (Cmpd 1.1, area %) | 0.15 |
| HPLC^{c} (total impurities, area %) | 0.45 |

^{a}SY = slightly yellow;
^{b}Amount terevalefim as percentage of initial amount added;
^{c}Measured using HPLC Method B of Example 7.

1. A composition comprising:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80); and
one or more aqueous components.

2. The composition of claim , wherein the composition comprises:
about 6 mg/mL to about 10 mg/mL terevalefim;
about 20% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

3. The composition of claim 1 or claim 2, wherein the composition comprises about 6 mg/mL terevalefim.

4. The composition of claim 1 or claim 2, wherein the composition comprises about 10 mg/mL terevalefim.

5. The composition of any one of the preceding claims, wherein the composition comprises about 40% (w/v) to about 60% (w/v) polyethylene glycol.

6. The composition of claim 5, wherein the composition comprises about 50% (w/v) polyethylene glycol.

7. The composition of any one of claims 1-4, wherein the composition comprises about 20% (w/v) to about 40% (w/v) polyethylene glycol.

8. The composition of claim 7, wherein the composition comprises about 30% (w/v) polyethylene glycol.

9. The composition of any one of the preceding claims, wherein the composition comprises about 5% (w/v) to about 15% (w/v) polysorbate.

10. The composition of claim 9, wherein the composition comprises about 10% (w/v) polysorbate.

11. The composition of claim 9, wherein the composition comprises about 6% (w/v) polysorbate.

12. The composition of any one of the preceding claims, wherein the aqueous components comprise aqueous buffer.

13. The composition of claim 12, wherein the aqueous buffer is aqueous phosphate buffer.

14. The composition of any one of the preceding claims, wherein the composition comprises:
about 10 mg/mL terevalefim;
about 50% (w/v) polyethylene glycol;
about 10% (w/v) polysorbate; and
one or more aqueous components selected from phosphate buffered saline and normal saline.

15. The composition of any one of claims 1-13, wherein the composition comprises:
about 6 mg/mL terevalefim;
about 30% (w/v) polyethylene glycol;
about 6% (w/v) polysorbate; and
one or more aqueous components selected from phosphate buffered saline and normal saline.

16. The composition of any one of claims 1-13, wherein the composition comprises:
about 6 mg/mL terevalefim;
about 30% (w/v) polyethylene glycol;
about 6% (w/v) polysorbate; and
aqueous buffer.

17. The composition of any one of the preceding claims, wherein the polyethylene glycol is polyethylene glycol 300.

18. The composition of any one of the preceding claims, wherein the polysorbate is polysorbate 80.

19. The composition of any one of the preceding claims, wherein the pH of the composition is between 7.5 and 7.9.

20. The composition of any one of the preceding claims, wherein the composition has been stored for at least 1 month.

21. The composition of any one of the preceding claims, wherein the composition has been stored for at least 6 months.

22. The composition of any one of the preceding claims, wherein the composition has been stored at about −20° C., about 5° C., or about 30° C.

23. The composition of any one of the preceding claims, wherein the composition has been stored at or below about 8° C.

24. The composition of any one of the preceding claims, wherein when the composition has been stored for at least 1 month, the pH of the composition is between 7.5 and 7.9.

25. The composition of any one of the preceding claims, wherein when the composition has been stored for at least 6 months, the pH of the composition is between 7.0 and 8.5.

26. The composition of any one of the preceding claims, wherein when the composition has been stored for at least 6 months, no more than 0.4 area % of compound 1.1: 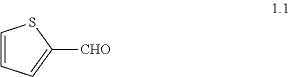
is detected by HPLC.

27. The composition of any one of the preceding claims, wherein when the composition has been stored for at least 1 month, no more than 0.1 area % of compound 1.1: 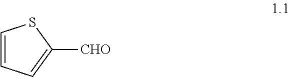
is detected by HPLC.

28. The composition of any one of the preceding claims, wherein the composition further comprises compound 3: 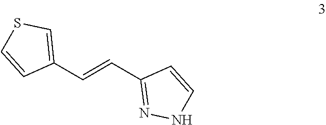 [SMILES: C(=C\c1cc[nH]n1)/c1ccsc1]
in an amount such that when a sample of the formulation is evaluated using HPLC, the area percent of compound 3 is between 0.05% and 1.0%.

29. The composition of any one of the preceding claims, wherein the composition has been sterilized.

30. A vial comprising the composition of any one of the preceding claims.

31. The vial of claim 30, wherein the vial comprises the composition in a total volume of about 20 mL to about 26 mL.

32. An article of manufacture comprising the composition of any one of claim 1-29 or the vial of claim 30 or 31; and a label comprising instructions for use.

33. A method comprising administering a composition of any one of claims 1-29 to a subject in need thereof.

34. The method of claim 33, wherein the composition is administered in an amount that provides a dose of 2 mg/kg terevalefim to the subject.

35. The method of claim 33 or claim 34, wherein the composition is administered once daily for three or four days.

36. The method of any one of claims 33-35, wherein the composition is administered intravenously.

37. The method of any one of claims 33-36, wherein the composition is administered intravenously as an infusion over about 30 minutes.

38. The method of any one of claims 33-37, wherein the method comprises steps of:
(i) providing a first composition comprising terevalefim;
(ii) diluting the first composition with normal saline to give a second composition comprising terevalefim; and
(iii) administering the second composition to the subject in need thereof.

39. The method of claim 38, wherein the step of diluting occurs under aseptic conditions.

40. The method of claim 38 or 39, wherein the second composition is administered to the subject within 1 day, 2 days, or 3 days of the step of diluting.

41. The method of any one of claims 38-40, wherein the first composition comprises:
about 10 mg/mL terevalefim;
about 40% (w/v) to about 60% (w/v) polyethylene glycol (e.g., polyethylene glycol 300);
about 5% (w/v) to about 15% (w/v) polysorbate (e.g., polysorbate 80); and
one or more aqueous components selected from phosphate buffered saline and normal saline.

42. The method of any one of claims 38-41, wherein the second composition comprises:
about 6 mg/mL terevalefim;
about 30% (w/v) polyethylene glycol (e.g., polyethylene glycol 300);
about 6% (w/v) polysorbate (e.g., polysorbate 80); and
one or more aqueous components selected from phosphate buffered saline and normal saline.

43. The method of any one of claims 33-42, wherein the total volume of composition administered per dose is less than about 100 mL.

44. A method comprising:
(i) providing terevalefim;
(ii) combining terevalefim with polysorbate and polyethylene glycol to give a mixture; and
(iii) adding one or more aqueous components to the mixture
to give a composition comprising terevalefim.

45. The method of claim 44, wherein the method comprises adding water and one or more phosphate salts to the mixture.

46. A method comprising:
(i) providing terevalefim;
(ii) combining terevalefim with polysorbate and polyethylene glycol to give a mixture; and
(iii) adding one or more aqueous components selected from phosphate buffered saline and normal saline to the mixture
to give a composition comprising terevalefim.

47. The method of any one of claims 44-46, wherein the composition is the composition of any one of claims 1-27.

48. The method of any one of claims 44-47, wherein the terevalefim is provided as crystalline Form A terevalefim.

49. The method of any one of claims 44-48, further comprising a step of determining homogeneity of the mixture and/or composition.

50. The method of any one of claims 44-49, further comprising a step of determining the pH of the composition.

51. The method of claim 50, further comprising a step of adjusting the pH of the composition if the determined pH is less than about 7.5 or greater than about 7.9.

52. The method of any one of claims 44-51, wherein the polyethylene glycol is polyethylene glycol 300.

53. The method of any one of claims 44-52, wherein the polysorbate is polysorbate 80.

54. A method comprising steps of:
(i) providing a compound of formula II-3: 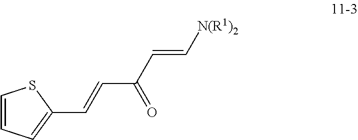
wherein each R¹ is independently C₁₋₆ aliphatic; and
(ii) contacting the compound of formula II-3 with hydrazine under suitable conditions to provide terevalefim.

55. The method of claim 54, wherein the compound of formula II-3 is compound 2.3:

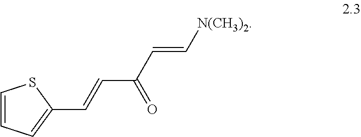 [SMILES: CN(C)/C=C/C(=O)/C=C/c1cccs1]

56. The method of claim 54 or 55, wherein between 1.0 and 1.5 molar equivalents of hydrazine hydrate are used.

57. The method of any one of claims 54-56, wherein the suitable conditions comprise acetic acid.

58. The method of any one of claims 54-57, further comprising a step of:
(i) providing a compound 2.1: 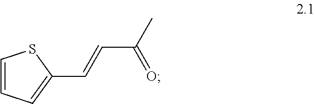
and
(ii) contacting compound 2.1 with a compound of formula II-2: 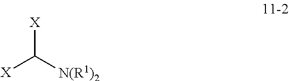
wherein each X is independently a suitable leaving group and each R¹ is independently C₁₋₆ aliphatic, under suitable conditions to provide the compound of formula II-3.

59. The method of claim 58, wherein the compound of formula II-2 is compound 2.2a:

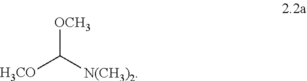

60. The method of any one of claims 54-59, further comprising a step of:
(i) providing compound 1.1: 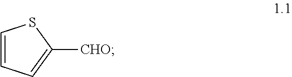
and
(ii) contacting compound 1.1 with acetone in the presence of a suitable base, to provide the compound 2.1.

61. The method of claim 60, wherein the suitable base is a hydroxide or alkoxide base.

62. The method of any one of claims 54-61, further comprising a step of recrystallizing the terevalefim from a suitable solvent.

63. The method of claim 62, wherein the terevalefim is recrystallized from acetonitrile and/or ethyl acetate.

64. A composition comprising terevalefim substantially free of acetonitrile.

65. A composition comprising terevalefim substantially free of hydrazine.

66. A composition comprising terevalefim substantially free of Impurity A.

67. The composition of any one of claims 64-66 prepared by the method of any one of claims 54-63.
